(19) United States
(12) Patent Application Publication
(10) Pub. No.: US 2025/0372248 A1
(43) Pub. Date: Dec. 4, 2025

(54) OPTIMIZATION AND PERSONALIZATION OF THERAPEUTIC PROTOCOLS

(71) Applicant: Roche Diabetes Care, Inc., Indianapolis, IN (US)

(72) Inventors: Tim Schwirtlich, Mannheim (DE); Andreas Kreuzer, Mannheim (DE); Chinmay Manohar, Indianapolis, IN (US); Sanjay Thakrar, Indianapolis, IN (US); Marina Rodenas, Meylan (FR)

(73) Assignee: Roche Diabetes Care, Inc., Indianapolis, IN (US)

(21) Appl. No.: 19/305,467

(22) Filed: Aug. 20, 2025

Related U.S. Application Data
(63) Continuation of application No. PCT/US2024/016386, filed on Feb. 19, 2024.
Application No. PCT/US2024/016386 is a continuation of application No. PCT/US2023/013418, filed on Feb. 20, 2023.

(51) Int. Cl. G16H 40/63

(52) U.S. Cl. CPC G16H 40/63

(57) ABSTRACT

Embodiments are described herein for optimizing and personalizing therapeutic protocols to treat a medical condition. An event of a therapeutic protocol may be determined. One or more triggering events may be detected for generating a notification associated with the event of the therapeutic protocol. The one or more triggering events may be determined to exceed a notification threshold indicating a level of confidence that a user will receive a notification transmitted in response to the one or more triggering events. The notification may be provided to a user to assist in treatment of a medical condition, such as a diabetic condition, in response to the detected triggering event associated with the event of the therapeutic protocol.

### BACKGROUND

Preset notifications used to carry out health and wellness related activities (e.g., diagnostic, medication dose, diet, exercise) are common features used to enhance compliance and/or adherence. However, in many cases and for a number of reasons, they are redundant, ignored and/or inconvenient to the user. Their inherent rigidity can result in the delay of desired health outcomes. In a particular case related to medication titration protocols (e.g., basal insulin titration), several events must be captured within a predetermined period of time in order to comply with the protocol and arrive at a therapeutically effective level of insulin. This includes measuring and recording blood glucose values at strict time frames in the morning (e.g., fasting blood glucose values) and administering and recording insulin doses in the evenings.

A person diagnosed with diabetes may typically be prescribed an insulin therapy to keep blood glucose levels within a targeted range. To begin insulin therapy, the person may follow strict therapeutic titration protocols (e.g., basal insulin titration) to reach a clinical target with the lowest possible dose, while mitigating diabetic symptoms and adverse side-effects of the insulin therapy. In order to comply with the therapeutic titration protocols and arrive at a therapeutically effective level of insulin, the person must capture several events within a predetermined period of time. For example, the person may be required to measure and record blood glucose values at strict time frames in the morning (e.g., fasting blood glucose values) and to administer and record insulin doses in the evenings. However, for a number of reasons, the person may ignore or fail to measure and record blood glucose values at the appropriate time frames. Additionally, in some cases, the person may discontinue or intermittently administer insulin, thereby putting the person at risk of becoming hypoglycemic or hyperglycemic depending on the person's blood glucose levels and insulin dosage. The inherent rigidity of these protocols fail to account for a patient's lifestyle. Moreover, if a prescriber is not aware of a patient's blood glucose levels or insulin dose levels during titration, the prescriber may not be able to adequately adjust titration levels. As such, achieving blood glucose levels within a targeted range may be delayed, or even not achieved. That is, if any of these events are not properly recorded, a titration protocol may be delayed and possibly terminated, thereby extending the time to achieve the proper medication levels and possibly advancing an already debilitating chronic disease.

### SUMMARY

Embodiments are described herein for optimizing and personalizing therapeutic protocols to treat a medical condition. As described herein, an event of a therapeutic protocol may be determined. The event may comprise a measurement event or a dosage event, for example. A triggering event may be detected for generating a notification associated with the event of the therapeutic protocol. The triggering event may include at least one of an activity that characterizes a circadian rhythm of the user, a device display triggering event, a device movement triggering event, a biometric triggering event, a time event, a period of time, a triggering event based on location information, or a triggering event based on calendar information, triggering events based on location information and calendar information. The notification may be provided to a user to assist in treatment of a medical condition, such as a diabetic condition, in response to the detected triggering event associated with the event of the therapeutic protocol.

On a condition that a response to the notification is received, management of the treatment of the diabetic condition may be performed. For example, the management of the treatment of the diabetic condition is performed by adjusting a scheduled time to monitor fasting blood glucose levels of the user; modifying a therapeutic dose of the therapeutic protocol; or adjusting a scheduled time to administer the therapeutic dose.

On a condition that the response to the notification fails to be received, the failure to respond to the notification for the event of the therapeutic protocol may be contextualized. The failure to respond may be contextualized based on calendar information, location information associated with a mobile device, and/or other triggering events (e.g., biometric triggering events, circadian rhythm triggering events, display device screen triggering events, and the like). The contextual information may indicate an inability of the user to respond to the notification.

A triggering event detection timeframe associated with the event of the therapeutic protocol may be activated, such that the triggering event is detected within the triggering event detection timeframe. The triggering event may be detected by detecting a keyword from at least one of a calendar event title, a calendar event location, calendar event description or calendar event metadata, for example, but not limited to, notification setting or availability status. The detected keyword may be compared to keywords associated with the notification. A timer may be adjusted corresponding to the notification based on the comparison of the detected keyword and the keywords associated with the notification. Adjusting the timer corresponding to the notification may include adjusting the timer of the notification to coincide with an occurrence of a calendar event associated with the at least one of the calendar event title or the calendar event location. Adjusting the timer corresponding to the notification may include contextualizing the triggering event based on the at least one of the calendar event title, the calendar event location, the calendar event description, or the calendar event metadata.

The therapeutic protocol may include an insulin titration protocol. The event of the insulin titration protocol may be a measurement event for measuring a fasting blood glucose level. It may be determined that the fasting blood glucose level is within a targeted therapeutic range and the notification may be provided to the user related to the blood glucose level being within the targeted therapeutic range. The event of the insulin titration protocol may be a dosing event for providing a dose of insulin. An optimal dose of insulin associated with the dosing event may be determined to achieve a targeted therapeutic range. The notification may be provided including the optimal dose of insulin to achieve the targeted therapeutic range.

### BRIEF DESCRIPTION OF THE DRAWINGS

FIG. 1 is a perspective view of a representative environment for optimizing and personalizing therapeutic protocols to treat a diabetic condition.

FIG. 2A shows a flowchart of an example process for managing therapeutic protocols.

FIG. 2B shows a flowchart of another example process for managing therapeutic protocols.

FIG. 3 shows a flowchart of an example process for titrating basal insulin.

FIG. 4 illustrates an example graphical user interface (GUI) that may display scheduling information to schedule a triggering event.

FIG. 5A shows a flowchart of an example process for detecting triggering events using different modalities.

FIG. 5B shows a flowchart of an example process for detecting triggering events from a user's calendar and a location of the user.

FIGS. 6A and 6B illustrate an example graphical user interface (GUI) that may display calendar information used to determine the location or an activity of a user.

FIGS. 7A and 7B illustrate example GUIs that include mealtime alerts that may be provided to a user.

FIG. 8 is a block diagram of an example computing device.

FIG. 9 is a block diagram of an example blood glucose monitoring device.

FIG. 10 is a block diagram of an example blood glucose meter (BGM) device.

### DETAILED DESCRIPTION

A relatively high percentage of patients are likely to end their insulin treatment during the first year of starting the insulin treatment. This may result from fears of insulin (act of injecting, adverse effect such as weight gain, etc.), fears of hypoglycemia causing the patient to maintain a low dose, a poor understanding of titration (e.g., changing dose, clear glycemic goals, etc.) causing the PwD to stay on a constant dose without adapting the dosage.

Diabetic patients (e.g., Type 2 diabetic patients) are mainly followed by their general practitioner as their HCP. These doctors are not diabetes specialists and yet they are the ones who realize many insulin initiations. However, the general practitioners may lack the time and/or specialization to give the necessary information to the patient regarding treatment. Moreover, patients admit to having difficulties mastering the injection technique and the gestures for monitoring blood sugar levels. General practitioners often express the difficulty of treating the diabetic condition, such as by carrying out insulin titrations because they are not specialized in diabetes. General practitioners are often afraid of hypoglycemia and, therefore, underestimate the dose of treatment and/or have difficulty getting the patient to adhere to their treatment.

People having medical conditions are often prescribed with medication that needs to be introduced in the body in effective doses for properly treating the medical condition, while limiting potential side effects. The effectiveness and potential side effects of a particular dose of medication may be difficult to determine for a particular person without introducing the medication to the body and performing appropriate testing. Drug titration is a process of adjusting a dosage of medication over time to identify how a particular individual's body will respond to the selected dosage. Therapeutic protocols, such as drug titration protocols, are established as a way to limit potential side effects, while adjusting and/or monitoring the effectiveness of the particular medication at the selected dosage over time. In many drug titration protocols, the selected dosage is started at a relatively low dose and the dosage is increased (e.g., up-titrated) to increase effectiveness until a target level of effectiveness is achieved (e.g., target dosage) without harmful side effects.

With regard to treatment of a diabetic condition, treatment may be performed with exercise, oral medicines, insulin and/or other injected medicines. After a person with diabetes (“PwD”) has been diagnosed, the PwD may be prescribed insulin (e.g., basal insulin) as a form of treatment. For example, basal insulin may be prescribed initially for a PwD with Type 1 diabetes or after exercise or other medication fails to treat the diabetic condition for a PwD with Type 2 diabetes. Basal insulin may act relatively slower than other types of insulin. The initially prescribed doses are often relatively small doses that are increased and/or monitored over the course of days or weeks according to a titration protocol to determine the effectiveness of the insulin for a particular individual and to limit potential side effects. When a person's body receives excess insulin in the bloodstream, the cells in the body may absorb too much glucose and can result in dangerously low glucose levels in the blood or hypoglycemia. With too little insulin in the body, the body can have difficulty moving glucose from the blood into the cells, causing high glucose levels in the blood or hyperglycemia. Adequate guidance and monitoring during the titration process can minimize adverse effects caused by basal insulin administration, while improving glycemic control in a timely manner.

In order to ensure proper titration to improve glycemic control, a PwD may follow a particular insulin titration protocol (e.g., basal insulin titration protocol). The particular insulin titration protocol may vary for different PwDs (e.g., depending on weight or other factors that may affect the effectiveness of the insulin). According to the particular titration protocol, PwDs may be required to take insulin at very specific times of day and/or days of the week for weeks at a time. For example, a PwD may measure their fasting blood sugar at a particular time of day and take a dose of insulin in a predefined number of units. The PwD may be required to re-test after one or more meals during the day. The PwD may be required to test in the evening, such as before going to bed. The testing may be performed for several days or weeks to determine whether the PwD is maintaining the proper insulin levels during the time period. The insulin titration protocol may require the PwD to increase the dosage one or more units each day or at a predefined number of days.

The insulin titration protocol may need to be rigidly adhered to in order to identify a proper target dosage. This may require a PwD to wake up at particular times of day or take insulin at times of day that are inconvenient. PwDs often forget to conform to the rigid schedule of the insulin titration protocol, because the titration process is often implemented on PwDs who are initially being introduced to insulin and are unfamiliar with taking insulin on a regular schedule. Additionally, PwDs often fail to take insulin or forget to take insulin when they are feeling well. However, in order to properly treat the diabetic condition of a PwD without harmful side effects, the insulin titration protocol should be adhered to as strictly as possible. Any failure to adhere strictly with the insulin titration protocol may result in delay of the desired health outcomes and benefits of the insulin, and/or harmful side effects. For example, a health care provider (“HCP”) may have reduced confidence to continue the insulin titration and even provide instructions to stop insulin titration if proper fasting blood glucose measurements are not taken and/or the injected insulin doses are not logged. With a lack of compliance to the titration protocols, the HCP that is monitoring the insulin titration may need further consultation with the PwD before restarting titration. Moreover, if there are no improvements, the HCP may consider other Oral Anti-diabetes Drugs (“OADs”). Further, by complying with the titration protocols, the HCP may provide other courses of treatment or modify the titration protocols (e.g., recommending the PwD take a meal-time boli, graduating the PwD to insulin pump therapy, prescribing another medication if the HCP determines there is an increased resistance to the prescribed insulin) if the PwD does not respond to the basal insulin therapy for various reasons.

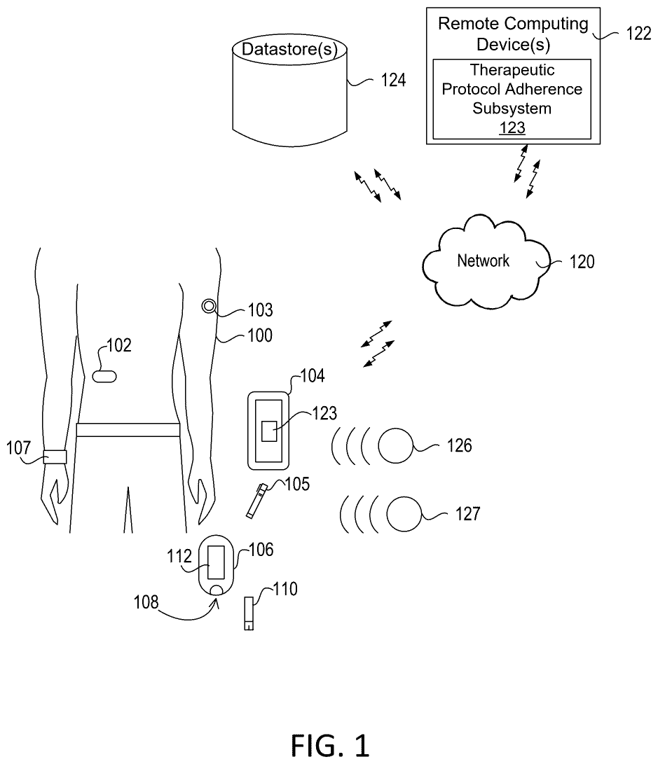

FIG. 1 is a perspective view of a representative environment for optimizing and personalizing therapeutic protocols to treat a medical condition, such as a diabetic condition. It is noted that the examples described herein optimize and personalize medication titration protocols (e.g., basal insulin titration protocols) to treat a diabetic condition; however, it is understood that the embodiments discussed herein are not limited to such and may relate to optimizing and personalizing therapeutic protocols to treat other medical conditions. For example, the embodiments discussed herein may relate to optimizing and personalizing titration protocols for blood thinners, anti-depressants, statins, anticonvulsants, sedatives, and the like.

As shown in FIG. 1, a user 100 with diabetes uses one or more blood glucose monitoring or treatment devices to help monitor or treat a diabetic condition, such as a metabolic syndrome, pre-diabetes, or Type 1/Type 2 diabetes. For example, the user 100 may use a blood glucose monitoring device to monitor blood glucose levels to initiate and/or optimize insulin therapy and achieve glycemic targets.

As used herein, the term “blood glucose monitoring device” refers to any device that detects and reports a level of glucose in the blood of the user, either through direct measurement of the blood or through an indirect detection process. A blood glucose level is also referred to as a blood sugar level. Examples of blood glucose monitoring devices include, but are not strictly limited to, continuous glucose monitoring devices, flash glucose monitoring devices, and blood glucose meters that provide a single measurement of blood glucose levels from a blood sample in a “spot” monitoring process. FIG. 1 depicts examples of blood glucose monitoring devices that are described in more detail below.

In some embodiments, the blood glucose monitoring device is a continuous glucose monitor (CGM) 102. The CGM 102 includes a subcutaneous sensor that is used to sense and monitor the amount of glucose in interstitial fluid of the user 100. The CGM 102 includes a transmitting device that is located directly over the sensor that wirelessly powers the data transfer from the sensor. The CGM 102 periodically communicates data indicating the blood glucose levels of the user 100 to an external device, such as a mobile device 104, for computing or storing the blood glucose levels of the user 100.

In some embodiments, the blood glucose treatment device is a pen device 105. The pen device 105 may include any injector pen capable of monitoring and/or managing insulin delivery. Example pen devices may communicate with an external device, such as the mobile device 104 and/or a remote computing device 122 executing a therapeutic protocol adherence subsystem 123, to determine insulin levels, calculate doses, track doses, deliver insulin, and/or provide other information such as notifications or alerts. In some examples, pen devices may periodically communicate data indicating the blood glucose levels of the user 100 to an external device, such as a mobile device 104, for computing or storing the blood glucose levels of the user 100.

As used herein, the term “mobile device” refers to any mobile electronic device that is capable of moving with a user as the user changes locations. Example mobile devices include mobile phones, smartphones, wearable devices, tablets, laptops, notebook computers, personal digital assistants (PDAs), and any other mobile electronic device that is capable of moving with a user. Some embodiments of the mobile device incorporate the blood glucose monitor into an integrated device.

Some embodiments of the mobile device 104 operate as a CGM controller device. Though the mobile device 104 is provided as an example of a device with which the CGM 102 communicates, the CGM 102 may communicate with other dedicated CGM controller devices for providing similar functionality that is described herein for the mobile device 104. In some cases, the CGM 102 processes the blood glucose data to provide an amount of glucose in interstitial fluid of the user 100. In some cases, the CGM 102 provides the blood glucose data to the mobile device 104 and/or pen device 105, and the mobile device 104 and/or pen device 105 processes the blood glucose data to manage the diabetic condition and provide treatment notifications as described herein.

In some embodiments, the blood glucose monitoring device is a flash glucose monitor (FGM) 103. The FGM 103 includes a subcutaneous sensor that is used to sense and monitor the amount of glucose in interstitial fluid of the user 100. A separate reader device, such as the mobile device 104, pen device 105, or another reader device, receives the blood glucose data from the sensor of the FGM 103 when the device is within range, such as but not limited to the RF range, of the sensor. The FGM 103 transmits an instantaneous blood glucose level or a graphical trend of the blood glucose level to the reader device for display. The mobile device 104 processes the blood glucose data to manage the diabetic condition and provide treatment notifications as described herein.

In some embodiments, the user 100 uses a blood glucose meter (BGM) 106 as a blood glucose monitoring device to monitor blood glucose levels. The BGM 106 includes a port 108 that receives a blood glucose measurement strip 110. The user 100 deposits a sample of blood on the blood glucose measurement strip 110. The BGM 106 analyzes the sample and measure the blood glucose level in the sample. The blood glucose level measured from the sample is displayed on a display 112 of the BGM 106 or communicated to an external device, such as the mobile device 104. The mobile device 104 processes the blood glucose data to manage the diabetic condition and provide treatment notifications as described herein.

The blood glucose level measured by the BGM 106 or computed using data received from the CGM 102 or FGM 103, is used to treat the diabetic condition of the user 100. The mobile device 104 communicates with the CGM 102, FGM 103, the BGM 106, and/or pen device 105 using wired or wireless communications. The mobile device 104, the CGM 102, a CGM controller, the BGM 106, the FGM 103, or pen device 105 are collectively referred to as user devices. The mobile device 104 communicates with the CGM 102, the FGM 103, the BGM 106, and/or pen device 105 using the same or different wireless protocols. For example, the mobile device 104 communicates with the CGM 102, FGM 103, the BGM 106, and/or pen device 105 using BLUETOOTH®, near field communication (NFC), THREAD®, WIFI®, ZIGBEE®, WI-MAX®, a cellular communication protocol, a proprietary wireless communication protocol, or another radio frequency (RF) communication protocol.

The mobile device 104 receives data and stores data for assisting in monitoring or treating the diabetic condition, such as, but not limited to optimizing and personalizing therapeutic protocols. The mobile device 104 receives input from the user 100 via a user interface being provided on a display. The mobile device 104 receives input via hard buttons or soft buttons provided on the display.

The mobile device 104 may be a connected smart device and/or may be in communication with other connected smart devices, such as the connected device 107, for example. Example connected smart devices may include a wearable device. The connected smart device may be an armband (e.g., a smart watch, such as an APPLE® watch, a FITBIT® armband, or other device capable of being worn on the arm of the user 100), a ring, glasses (e.g., GOOGLE® GLASS™), a headset (e.g., BLUETOOTH® headset), clothing (e.g., shirts, gloves, etc.), or another wearable device capable of being worn by the user 100. The connected smart device may be a wearable device or other device capable of monitoring the heartrate of the user 100, such as a heart rate monitor. The connected smart device may include a device capable of monitoring a wake/sleep state of the user, such as a wearable device, a heart rate monitor, a smart bed, or another device.

The mobile device 104 is configured to determine information corresponding to the device's location (i.e., location information). For example, the mobile device 104 is able to determine the geolocation (e.g., latitude and longitude) of the mobile device 104 using signals from a global positioning system (GPS) or triangulation via cellular communications. In some cases, the mobile device 104 determines a relative location of the mobile device 104 via an indoor positioning technique by locating anchor nodes, such as WiFi access points, an internet of things (IOT) device 127, and/or an RF beacon device 126, within a location that are communicably coupled to the mobile device 104. The RF beacon device 126 communicates a unique identifier via a short-range wireless communication, such as a BLUETOOTH® low energy (BLE) beacon or an NFC beacon. The mobile device 104 receives the RF beacon and performs a lookup in a database (e.g., in information from the datastores 124) to determine a relative location associated with the unique identifier. For example, the mobile device 104 determines that the RF beacon indicates that the device is in a particular room in a home or building, on a certain floor in a building, close to a predefined object, or is within the RF range of a beacon associated with another object or location. In some cases, the mobile device 104 determines a relative location using IOT device 127. The IOT device 127 may be configured to be associated with a location, such as, but not limited to, a room in a user's home. In some cases, when the IOT device 127 is within range of the mobile device 104, the IOT device 127 communicates the relative location associated with the IOT device 127.

Some embodiments of the mobile device 104 include one or more sensors for detecting a relative position of the device or information about the user 100. The mobile device 104 detects a movement or a change in orientation. Based on the movement or change in orientation (or lack thereof) of the mobile device 104 over a period of time, the mobile device 104 detects that the user 100 is standing, sitting, or lying down. In other words, the mobile device 104 detects/infers that the user 100 is awake. The mobile device 104 detects that the user 100 is exercising when the movement or a change in orientation is greater than a threshold for a period of time. The mobile device 104 detects the heartrate of the user 100 using a heartrate sensor. Based on the heartrate and the movement of the user 100 over a period of time, the mobile device 104 detects whether the user 100 is asleep or awake. The information about the mobile device 104 or the user 100 is used to provide information about or treat the diabetic condition.

The mobile device 104 provides information to the user 100 about the user's diabetic condition. For example, the mobile device 104 provides blood glucose levels, provides meal-related information, provides exercise-related information, provides treatment notifications, or generates graphs and other graphical user interfaces for display, or generates notifications that are provided to the user 100. The mobile device 104 provides therapeutic protocol data to the pen device 105. For example, the mobile device 104 provides insulin dose levels for an associated medication titration event (e.g., basal insulin titration event) to the pen device 105. Having received the insulin dose level, the pen device 105 is configured to administer a corresponding amount of insulin.

The mobile device 104 communicates with other devices directly via a wired communication or a short-range wireless communication (e.g., WI-FI®, BLUETOOTH®, BLE, NFC, or another suitable short-range wireless communication). The mobile device 104 communicates indirectly with remote computing device(s) 122, or datastore(s) 124 via a network 120 (e.g., using a WI-FI® network, a cellular network, a WI-MAX® network, or another wired or wireless network). The network 120 is a wired or wireless network. The network 120 is used to communicate over the Internet to other devices.

The mobile device 104 communicates with the remote computing device(s) 122 to generate user interfaces for display on the mobile device 104, perform remote computation, or to otherwise control a remote computing device. For example, the mobile device 104 provides a user interface via an application (e.g., a web browser or other local application) that is generated locally for providing access to locally stored data or data from a remote computing device 122. The mobile device 104 generates instructions for optimizing and personalizing therapeutic protocols, such as, for example, modifying an insulin titration protocol via remote computing devices 122 based on information received from the user 100, the CGM 102, the FGM 103, or the BGM 106. Example remote computing device(s) 122 to which the mobile device 104 sends communications for optimizing and personalizing therapeutic protocols include a remote computer (e.g., a server, a laptop, or other computer), an external speaker, an external display device (e.g., television, monitor, or another device having an external display), or another remote computing device.

The therapeutic protocol adherence subsystem 123 operates on the remote computing device(s) 122, and may be utilized by one or more devices, such as the mobile device 104 and the pen device 105, via an application downloaded from the remote computing device(s) 122 or a third-party application store, and executed on the mobile device 104. The therapeutic protocol adherence subsystem 123 may be a software-based program, downloaded from the remote computing device(s) 122, and installed on one or more devices, such as the mobile device 104. The therapeutic protocol adherence subsystem 123 may be utilized as a software service provided by a third-party cloud service provider (not shown). In one or more cases, the therapeutic protocol adherence subsystem 123 may be implemented as hardware, software, or a combination of both hardware and software. The therapeutic protocol adherence subsystem 123 may be operable at a single device or may be distributed across multiple devices (e.g., mobile device 104 and/or remote computing device 122). The therapeutic protocol adherence subsystem 123 may be stored on a removable storage device, such as a USB flash drive, and downloaded onto the remote computing device(s) 122, and/or mobile device 104. The therapeutic protocol adherence subsystem 123 may be executed to implement one or more of the processes, such as modifying therapeutic protocols and/or detecting triggering events, as described herein.

The therapeutic protocol adherence subsystem 123 communicates with the datastores 124 to store information or retrieve information. The information includes information related to the user 100, the CGM 102, the FGM 103, the BGM 106, or the pen device 105. For example, the therapeutic protocol adherence subsystem 123 receives treatment information, via the mobile device 104, associated with the user 100 as input or receive blood glucose information from the CGM 102 or the BGM 106 and send the information to the datastore(s) 124 via the network 120. Stored information is retrieved from the datastore(s) 124 for treatment of the diabetic condition of the user 100. For example, the therapeutic protocol adherence subsystem 123 retrieves an amount of insulin delivered to the user 100 or corresponding times of delivery. In another example, the therapeutic protocol adherence subsystem 123 retrieves notifications associated with a therapeutic protocol. In yet other examples, the therapeutic protocol adherence subsystem 123 retrieves location information, calendar event information, biometric triggering information, circadian rhythm triggering information, and/or other triggering information from the datastore(s) 124. In one or more cases, calendar information and/or location information may be provided and stored to the datastore(s) 124 via third party services, and may be accessible by the therapeutic protocol adherence subsystem 123. The datastore(s) 124 include one or more remote storage locations, which are collectively referred to as cloud storage. For example, the datastore(s) 124 store information regarding one or more personal characteristics of the user 100 (e.g., the user's age or gender), calendar events, therapeutic protocols associated with the user 100.

One or more of the user devices are implemented to provide the user 100 with assistance in carrying out health and wellness related activities, such as diagnostics and/or treatment of medical conditions. The user devices may be used to enhance compliance and/or adherence to particular health-related protocols to assist in treatment of a particular medical condition. In the case of drug titration protocols, several events need to be captured within a predetermined period of time in order to comply with the protocol and arrive at a therapeutically effective level of medicine for proper treatment of a medical condition. With regard to treatment of a diabetic condition, compliance with basal insulin titration protocols should be strictly adhered to in order to properly treat the diabetic condition without the harmful side effects described herein.

The user devices described herein may be used to adhere to drug titration protocols, such as insulin titration protocols. The user devices may provide notifications to help manage the diabetic condition according to the insulin titration protocols. The titration protocols and notifications are used to enhance user compliance with therapeutic protocols to treat a medical condition. To better enhance user compliance, therapeutic protocols, such as titration protocols, can be modified based on user input, lack of user input, user-specific characteristics, or the user environment, as described herein.

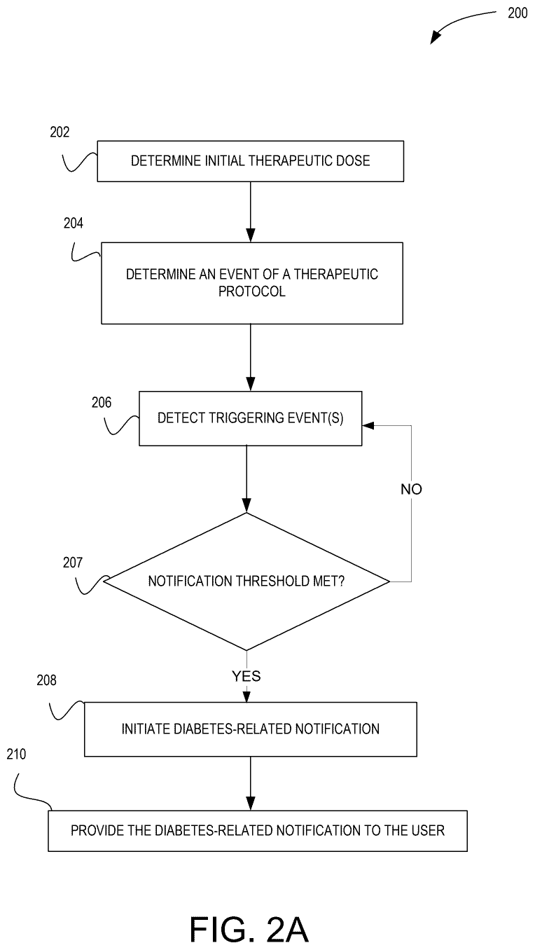

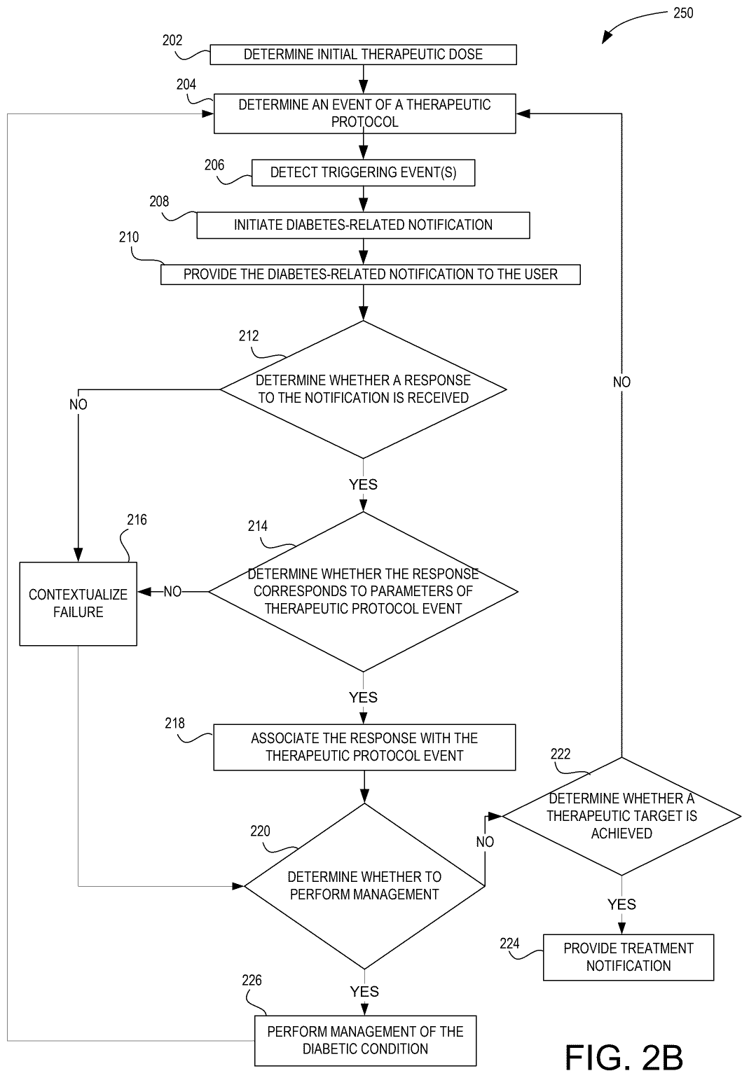

FIG. 2A shows a flowchart of an example process 200 for managing adherence to therapeutic protocols, such as, but not limited to, a basal insulin titration protocol. One or more portions of the process 200 may be performed by one or more computing devices. For example, the one or more portions of the process 200 may be performed by one or more mobile devices, such as mobile device 104 shown in FIG. 1, and/or one or more remote computing devices, such as remote computing device 122 shown in FIG. 1. One or more portions of the process 200 may be stored in memory as computer-readable or machine-readable instructions that may be executed by a processor of the one or more computing devices. One or more portions of the process 200 may be performed by one or more subsystems operating on one or more computing devices. For example, one or more portions of the process 200 may be performed by one or more devices operating a therapeutic protocol adherence subsystem, such as the therapeutic protocol adherence subsystem 123 shown in FIG. 1. Though portions of the process 200 may be described herein as being performed by a therapeutic protocol adherence subsystem operating on a particular computing device, the process 200 may be performed by another computing device or distributed across multiple computing devices, such as one or more mobile devices and/or remote computing devices.

As illustrated in FIG. 2A, the therapeutic protocol adherence subsystem may determine an initial therapeutic dose at 202 for initializing medication titration protocols (e.g., basal insulin titration). For example, a mobile device 104 may launch an application providing access to the therapeutic protocol adherence subsystem 123, via a GUI displayed on the mobile device 104, and initialize the medication titration protocol. The mobile device 104, via the therapeutic protocol adherence subsystem 123, may obtain the initial therapeutic dose from a variety of sources to begin titration. In some cases, a prescriber may provide a starting therapeutic dose to the therapeutic protocol adherence subsystem 123 via the remote computing device(s) 122. In some cases, the user 100 may input the starting therapeutic dose into the therapeutic protocol adherence subsystem 123, via a GUI displayed on the mobile device 104. In some cases, the therapeutic protocol adherence subsystem 123 may retrieve a starting therapeutic dose from the datastore(s) 124. In one example, the starting therapeutic dose for an insulin injection, such as Lantus®, may be 10 units of basal insulin per day or a weight-based dose of 0.2 units/kg/day. In another example, the starting therapeutic dose for an insulin injection, such as Levemir®, may be 10 units of basal insulin per day or a weight-based dose of 0.1-0.2 units/kg/day. In other examples, the starting therapeutic dose may be based on the weight and a measured fasting glucose level of the user 100. To begin basal insulin titration, the user 100 or another person, such as a nurse or physician, subcutaneously injects the starting basal insulin dose into the user 100.

Having determined the initial therapeutic dose, the therapeutic protocol adherence subsystem 123 may determine an event of a therapeutic protocol at 204. An event of a therapeutic protocol may be, for example, a step of a titration process. The steps in the titration process may include measurement events and/or insulin dosing events. For example, the insulin dosing events may occur once a day or multiple times a day. Each insulin dosing event may include a corresponding dosage in units. The measurement events may include a fasting measurement event for measuring fasting blood glucose values, mealtime measurement events for measuring blood glucose values before/after a meal, and/or bedtime blood glucose measurement events for measuring blood glucose values prior to bedtime. The therapeutic protocol may be implemented with one or more events over a period of time to administer the therapy to achieve a therapeutic target. The therapeutic target may be a target dosage and/or target blood glucose level. An event of a therapeutic protocol may be, for example, a step of a titration process.

Each event of the therapeutic protocol may be stored with corresponding parameters for successful performance of the event. The parameters may include timing or a time frame within which event is to be performed according to the therapeutic protocol. The parameters for dosing events may include a dosage amount in units. The parameters for measurement events may include measurement values/ranges within which glucose measurement results are to be within for measurement events.

Each event of the therapeutic protocol may be stored with corresponding triggering events for triggering notifications to the user 100. The notifications may include one or more of the parameters for the corresponding event, such as the dosage amounts and/or the measurement values/ranges. As illustrated in FIG. 2A, one or more triggering events are detected by the therapeutic protocol adherence subsystem 123 at 206 for triggering a notification for the event of the therapeutic protocol. The triggering event may include a location-based triggering event based on location information, device display triggering event, a device movement triggering event, a biometric triggering event, a time event, a period of time, triggering events based on calendar information, triggering events based on location information and calendar information, or a combination of the aforementioned.

The location-based triggering event may include a geolocation or other relative location of the mobile device 104 within a space based on location information associated with the mobile device 104. The location-based triggering event may be triggered in response to the location of the mobile device being within a geofence of a space, such as a room or building for example. The location information may be obtained from the location of the mobile device 104 and/or another connected device 107, such as a location of the mobile device 104 as a set of latitude and longitude coordinates from GPS data. The location-based triggering event may be triggered based on a keyword analysis. For example, the therapeutic protocol adherence subsystem 123 may perform keyword analysis of the calendar information to identify a location of a calendar event (e.g., the keyword “Cafe,” “Gym,” “Work,” “Home,” or other location in a location field of the calendar event). The location may include a street address, which the therapeutic protocol adherence subsystem 123 may attempt to match the street address to a known entity (e.g., a business, residence, or other entity).

The device display triggering event may be triggered by identification of an instance at which a display of the mobile device 104 turns on. The operating system of the mobile device 104 may have the capability to track such a trigger that may be provided to the therapeutic protocol adherence subsystem 123 via an application programming interface (API) (e.g., an ACTION_SCREEN_ON event provided in the ANDROID API). The device display event trigger may be detected by the mobile device 104 identifying a first instance of the power to the display turning on or an intensity of the display increasing after a predefined period of time. The device display event trigger may indicate that the user 100 is possibly interacting with the mobile device 104 and/or is capable of engaging in the dosing events and/or measurement events of the titration protocol.

The device movement triggering event may indicate a movement of the mobile device 104 or a first movement of the mobile device 104 within a predefined period of time. The mobile device 104 may track the movement via an internal sensor, such as an accelerometer or a gyroscope and the therapeutic protocol adherence subsystem 123 may detect the device movement triggering event and interpret the device movement triggering event as an indication of a first interaction by the user 100 with the mobile device 104 within a predefined period of time (e.g., a first daily interaction).

The biometric triggering event may be detected in response to a biometric parameter of the user 100. The biometric parameter may be tracked by a connected smart device, such as the mobile device 104 or the connected smart device 107. The biometric parameter may indicate a wake state or sleep state of the user 100. The heart rate of the user may be accessible via the remote computing device 122 and/or the datastore 124. For example, the heart rate of the user may be accessed by the therapeutic protocol adherence subsystem 123 indirectly over cloud services offered by third-party records offered by another service or application (e.g., Apple Health records), which is active in the background of the mobile device 104 and observes wake-up patterns via measured and sustained increases in heart rate.

The time event or period of time for the triggering event may be determined based on day/night activity levels. For example, the triggering events may be defined differently for people that work during different shifts (e.g., during the day vs. during the night). The time and/or periods of time may be adjusted based on user input or automatically (e.g., from morning/evening to evening/morning or even morning/morning). For example, if the user 100 misses a predefined number of notifications or has difficulties adjusting the therapy to a specific lifestyle (e.g., extended titration cycles, missed doses, etc.), then the therapeutic protocol adherence subsystem 123 may display a notification to the user 100 and/or a healthcare provider system proposing another protocol or schedule. The user 100's schedule may be dynamically tracked and/or updated by tracking when the user 100 is asleep to identify a time or period of time for fasting within the titration protocol.

As described herein, each event of the therapeutic protocol (e.g., dosing event or measurement event) may have one or more corresponding triggering events. Multiple triggering events may be detected for triggering a notification (e.g., diabetes-related notification). For example, each event of the therapeutic protocol may be stored with an activated triggering event detection time period within which other triggering events may be detected for triggering a diabetes-related notification to the user 100. The activated triggering event detection time period for a given event of the therapeutic protocol may define the time period within which other events may be detected for triggering notifications related to that particular event of the therapeutic protocol.

At 207, the protocol adherence subsystem 123 may determine whether the notification threshold has been met. For example, the notification threshold may be met when one or more triggering events are detected. If the notification threshold has not been met by one or more triggering events, then the protocol adherence subsystem 123 may detect additional triggering events until the notification threshold is met at 207. The number and/or type of triggering events may be predetermined for providing a corresponding diabetes-related notification. In an example, a first triggering event may be detected at 206 and/or one or more triggering events may be detected to confirm that the diabetes-related notification should be initiated. After the one or more additional triggering events are detected, the notification threshold may be determined to be met at 207. For example, the protocol adherence subsystem 123 may detect that a location-based triggering event is met and then detect a device movement triggering event or a device display triggering event to increase the confidence level that the user 100 is at their mobile device 104 for providing the notification. The notification threshold may be increased to require a greater confidence that the diabetes-related notification will be received by the user 100.

Different triggering events may be weighted and the notification threshold may be set to a certain weight. Each triggering event may be weighted the same or differently. For example, a location-based triggering event may be given a greater weight than triggering events based on calendar information. The notification threshold may be increased to a greater weight to require a greater confidence The notification threshold may be increased to require a greater confidence that the diabetes-related notification will be received by the user 100.

The protocol adherence subsystem 123 and/or the user 100 may adjust the notification threshold and/or weights associated with different triggering events. For example, the protocol adherence subsystem 123 may include intelligence that enables the protocol adherence subsystem 123 to determine the relative influence of different triggering events on obtaining a response by the user 100 to a notification and assign a relative weight to the triggering events.

After the notification threshold is met at 207, the protocol adherence subsystem 123 initiates a diabetes-related notification at 208. The protocol adherence subsystem 123 may initiate the diabetes-related notification by setting a timer to provide the diabetes-related notification. The protocol adherence subsystem 123 may set the timer to display the diabetes-related notification via a GUI associated with the therapeutic protocol adherence subsystem 123. The protocol adherence subsystem 123 may initiate the timer of the diabetes-related notification to occur when a triggering event is detected. For example, the protocol adherence subsystem 123 may schedule the diabetes-related notification to occur upon detecting the triggering event (e.g., in real-time or near real-time). The protocol adherence subsystem 123 may initiate the timer to cause the diabetes-related notification to occur at a time period different than when the triggering event is detected. For example, the protocol adherence subsystem 123 may schedule the diabetes-related notification to occur at a time earlier than the triggering event, as described herein. In another example, the protocol adherence subsystem 123 may delay or reschedule the diabetes-related notification to occur after detecting the triggering event, as described herein.

The protocol adherence subsystem 123 provides the diabetes-related notification to the user at 210. The notification may be a visual notification and/or an audible notification. For example, the protocol adherence subsystem 123 may cause the mobile device 104 and/or the other connected device 107 to display the diabetes-related notification via a GUI and/or provide an audible alert via a speaker.

FIG. 2B shows a flowchart of an example process 250 for managing adherence to therapeutic protocols, such as, but not limited to, a basal insulin titration protocol. One or more portions of the process 250 may be performed by one or more computing devices. For example, the one or more portions of the process 250 may be performed by one or more mobile devices, such as mobile device 104 shown in FIG. 1, and/or one or more remote computing devices, such as remote computing device 122 shown in FIG. 1. One or more portions of the process 250 may be stored in memory as computer-readable or machine-readable instructions that may be executed by a processor of the one or more computing devices. One or more portions of the process 250 may be performed by one or more subsystems operating on one or more computing devices. For example, one or more portions of the process 250 may be performed by one or more devices operating a therapeutic protocol adherence subsystem, such as the therapeutic protocol adherence subsystem 123 shown in FIG. 1. Though portions of the process 250 may be described herein as being performed by a therapeutic protocol adherence subsystem operating on a particular computing device, the process 250 may be performed by another computing device or distributed across multiple computing devices, such as one or more mobile devices and/or remote computing devices.

As illustrated in FIG. 2B, the process 250 may include one or more similar steps to the process 200 shown in FIG. 2A. For example, the process 250 may include one or more of steps 202-210. Though not separately shown in FIG. 2B, the process 250 may determine whether a notification threshold is met based on the detected triggering events at 206. For example, the step 206 may detect one or more triggering events until a notification threshold is met and the notification threshold may be implemented as described elsewhere herein.

The process 250 includes additional steps based on whether a response to the notification is received from the user. For example, after the diabetes-related notification is provided to the user 100 at 210, the protocol adherence subsystem 123 determines whether a response to the diabetes-related notification is received at 212. The protocol adherence subsystem 123 determines that a response to the diabetes-related notification is received based on receiving an indication of a user input via a GUI being displayed on the mobile device 104 and/or another connected device 107 via the therapeutic protocol adherence subsystem 123. For example, the user 100 may provide a response to the diabetes-related notification via one or more buttons or links displayed on the associated GUI. The buttons or links may allow the user 100 to provide input in response to the diabetes-related notification to indicate whether the user performed the event of the therapeutic protocol (e.g., dosing event or measurement event).

For the cases in which the therapeutic protocol adherence subsystem 123 determines that a response to the diabetes-related notification is not received at 212 (e.g., after a predefined period of time), the therapeutic protocol adherence subsystem 123 contextualizes the failure to respond to the triggering event at 216 to determine why a response to the notification failed to be received. By contextualizing the triggering event, the therapeutic protocol adherence subsystem 123 may determine a reason for non-compliance with the therapeutic protocol event or failing to provide a response to the diabetes-related notification.

In some cases, the therapeutic protocol adherence subsystem 123 contextualizes the failure to respond to the notification to determine a causative pattern of non-compliance based on calendar events and/or location information. Non-compliance with scheduled events (e.g., dosing events and/or measurement events) may be remedied by the therapeutic protocol adherence subsystem 123 (e.g., before a healthcare provider intervenes). For example, if a diabetes-related notification (e.g., a request to perform a dosing event or measuring event, such as a request to measure fasting blood glucose levels) is scheduled to occur at 7:00 am each morning, the therapeutic protocol adherence subsystem 123 may review calendar information that may be received from a remote computing system, such as the remote computing device 122, or calendar information accessible from a datastore, such as datastore 124. The therapeutic protocol adherence subsystem 123 may review events in the calendar information at the same time or within a same period of time scheduled for the event of the therapeutic protocol and/or the triggering events associated therewith to determine if an overlapping or adjacent event may be interfering with the scheduled events of the therapeutic protocol. If the protocol adherence subsystem 123 determines that a response to the notification for the user to measure the blood glucose measurement at 7:00 am failed to be received (e.g., within a predefined period of time), then the therapeutic protocol adherence subsystem 123 determines that the user failed to comply with the event of the therapeutic protocol due to the calendar event.

The therapeutic protocol adherence subsystem 123 may contextualize the failure to respond at 216 using location information. The location information may include a location of the calendar event in the calendar information or a location of the mobile device 104 or other connected device 107. For example, the event title is “Breakfast with John” and the event location is “Cafe”. The therapeutic protocol adherence subsystem 123 may perform keyword analysis of the calendar information to identify a location of a calendar event (e.g., the keyword “Cafe,” “Gym,” “Work,” “Home,” or other location in a location field of the calendar event). The location may include a street address, which the therapeutic protocol adherence subsystem 123 may attempt to match the street address to a known entity (e.g., a business, residence, or other entity). The location information may be obtained from the location of the mobile device 104 and/or another connected device 107, such as a location of the mobile device 104 as a set of latitude and longitude coordinates from GPS data. The therapeutic protocol adherence subsystem 123 may be configured to read and interpret the location information via a referential database or other architecture that associates keywords with functional logic. The therapeutic protocol adherence subsystem 123 may use calls to a location services API to obtain the location information.

The accessed location information may indicate that the user 100 is at a location that may cause the user 100 to be away from their mobile device 104 (e.g., gym, work, etc.) or may have their notifications silenced (e.g., theater, work, etc.). The accessed location information may suggest that the user 100 is traveling and unable to access their mobile device 104. The therapeutic protocol adherence subsystem 123 may choose to adjust the time associated with the event based on the location information or based on updates to the location information (e.g., change in location or location is stationary for predefined period of time). The therapeutic protocol adherence subsystem 123 may send a notification to another connected device, such as the connected device 107, that may be physically on the user to increase the chance of user 100's awareness and adherence to the event in the therapeutic protocol.

The therapeutic protocol adherence subsystem 123 may store the results of the calendar analysis, location analysis, and/or other results of analysis performed to contextualize the failure to respond to the notification as a non-compliant event. The therapeutic protocol adherence subsystem 123 may assume that a failure to respond to the notification indicates a failure to perform the event of the therapeutic protocol. The therapeutic protocol adherence subsystem 123 may associate the non-compliant events related to the same or similar diabetes-related notifications (e.g., a request to perform a dosing event or measuring event, such as a request to measure fasting blood glucose levels). In one or more cases, the therapeutic protocol adherence subsystem 123 may determine a pattern develops if multiple non-compliant events corresponding to the same or similar diabetes-related notifications are logged (e.g., multiple times a day, or multiple days a week) and the same adjacent or overlapping calendar events are present in the times or time periods during which the non-compliant events are to be performed in the therapeutic protocol.

To avoid the adjacent or overlapping calendar events, the therapeutic protocol adherence subsystem 123 may adjust the time or time period of the diabetes-related notification by a predetermined buffer of time to occur either before or after the potentially conflicting event. In one example, therapeutic protocol adherence subsystem 123 may adjust the time or time period of the diabetes-related notification when multiple non-compliant events corresponding to the same or similar diabetes-related notifications are logged. In some cases, before adjusting the timer of the diabetes-related notification, the therapeutic protocol adherence subsystem 123 may provide a notification, via a GUI associated with the therapeutic protocol adherence subsystem 123, to the user 100 that a potential conflict has been detected. The therapeutic protocol adherence subsystem 123 may provide the notification with an inquiry as to whether the non-compliance is associated with the potentially conflicting calendar event. For example, the therapeutic protocol adherence subsystem 123 may provide a notification such as “We noticed that you missed logging your fasting blood glucose twice this week around the times that you scheduled your ‘morning run’. Would you like for us to set the fasting measurement notification 15 minutes earlier?”.

Based on the contextual information, the time or time period for a notification for an event in the therapeutic protocol may be moved ahead in predefined intervals. The notification may be moved until the contextual information (e.g., calendar information, location information, or other information) changes and/or indicates that the user 100 is free and/or in a location at which the event may be performed. Different locations may be identified for being able to perform a corresponding event in the therapeutic protocol. The therapeutic protocol adherence subsystem 123 can be configured to allow the user 100 to confirm such changes to notification times when they are detected (e.g., active changes) or allow the system to automatically make them without notification and confirmation (e.g., passive changes).

The therapeutic protocol adherence subsystem 123 may adjust a tolerance level associated with the notifications and/or activity logging events when contextualizing the failure to respond within the timeframe defined by the therapeutic protocol at 216. For example, the therapeutic protocol adherence subsystem 123 may identify a predefined period of time to allow for tolerance for receiving the response to the notification or otherwise receive an indication that the event related to the protocol (e.g., dosing event or measurement event) has been performed. The therapeutic protocol adherence subsystem 123 may identify the calendar event by analyzing the calendar information and allow for a tolerance level that is a predefined period of time before and/or after the time for the diabetes-related notification or a predefined period of time before and/or after the calendar event. For example, the therapeutic protocol adherence subsystem 123 may identify a meeting time in the calendar information or identify that the user 100 is traveling and allow for a greater level of tolerance by allowing for a response within a predetermined period of time before and/or after the meeting time or the notification time. The therapeutic protocol adherence subsystem 123 may identify particular meetings or keywords in the calendar information and allow for a tolerance by allowing for a response within a predetermined period of time before and/or after the event having the predefined keyword (e.g., lunch, meeting, wake-up, travel, etc.). The therapeutic protocol adherence subsystem 123 may allow for a greater tolerance level at predefined times of day and/or times of week. For example, the therapeutic protocol adherence subsystem 123 may allow for a predefined period of time for the user 100 to provide a response to the notification on weekends (e.g., windows of acceptable activity logging extended by 30 minutes).

In contextualizing the failure to respond at 216, the therapeutic protocol adherence subsystem 123 may also or alternatively use sleep/wake settings within the operating system of the mobile device 104. For example, the sleep/wake settings of the operating system may be used when the calendar information is unavailable. The therapeutic protocol adherence subsystem 123 may use the sleep/wake settings within the operating system of the mobile device 104 to determine and set one or more events at or within a predefined period of time before or after a sleep/wake time set at the mobile device 104. Sleep/wake settings may refer to specific settings that user sets within the operating system, for example, by enabling Do Not Disturb or Bedtime modes for a certain time period (e.g., 10 PM to 7 AM) to silence notifications, auto-decline incoming calls, and the like.

For the cases in which the therapeutic protocol adherence subsystem 123 determines that a response to the notification is received at 212, the therapeutic protocol adherence subsystem 123 determines whether the response corresponds to the parameters of the therapeutic protocol event at 214. For example, therapeutic protocol adherence subsystem 123 may determine at 214 whether the response was received at the time or within the timeframe indicated by the parameters of the corresponding event. The therapeutic protocol adherence subsystem 123 may receive an indication of the dosage amount for a dosing event and/or measurements associated with the measurement events and determine whether the dosage amount or the measurement values/ranges correspond to the parameters of the event. The therapeutic protocol adherence subsystem 123 may determine that a received measurement for a fasting blood glucose level corresponds to the time of the therapeutic protocol event (e.g., determining a fasting blood glucose level during the morning before the user eats or within predetermined period of time in the morning). In an example, the therapeutic protocol adherence subsystem 123 may receive an indication that the user administered an injection of a basal insulin dose of 10 units at 9:00 PM and determine that the indication corresponds to the appropriate time period and dosage for performance of therapeutic protocol event (e.g., administer an injection of basal insulin at a predetermined dose within a predefined period of time of day). In another example, a therapeutic protocol event may correspond to administering an injection of basal insulin at a dose of 12 units at 9:30 PM. However, the therapeutic protocol adherence subsystem 123 may receive an indication that the user administered an injection of a basal insulin dose of 10 units at 11:00 PM, and therefore determines that the response does not correspond to the timing and/or appropriate dosage for the therapeutic protocol event. For the cases in which the therapeutic protocol adherence subsystem 123 determines that the response corresponds to an event of the therapeutic protocol, the therapeutic protocol adherence subsystem 123 associates the response with the therapeutic protocol event at 218. The therapeutic protocol adherence subsystem 123 may store the provided response with the associated therapeutic protocol event.

For the cases in which the therapeutic protocol adherence subsystem 123 determines that the response does not include information corresponding to the parameters of the therapeutic protocol event, the therapeutic protocol adherence subsystem 123 contextualizes the failure to conform to the therapeutic protocol at 216. For example, the therapeutic protocol adherence subsystem 123 may contextualize the failure to respond to the notification within the timeframe by analyzing calendar information, location information, and/or other contextual information to determine if an overlapping or adjacent event is interfering with the user from providing an adequate response that corresponds to the therapeutic protocol event. The therapeutic protocol adherence subsystem 123 may analyze the calendar event information and determine that although the user administered an injection of basal insulin an overlapping or adjacent event interfered with the user from administering the injection at the requested time in the therapeutic protocol. The therapeutic protocol adherence subsystem 123 may analyze the dosage information provided for a dosing event and/or measurement values/ranges for a measurement event to determine whether the parameters of the corresponding event have been met. Further, to contextualize failure, the therapeutic protocol adherence subsystem 123 may provide a notification as to the reason the user failed the corresponding event. For example, the therapeutic protocol adherence subsystem 123 may provide a notification such as “We noticed that you administered a lower dose of insulin than the scheduled dose. Please provide comments as to why you did not administer to scheduled dose.”

At 220, the therapeutic protocol adherence subsystem 123 determines whether to perform management of the diabetic condition. In some cases, the therapeutic protocol adherence subsystem 123 determines whether to perform management of the diabetic condition based on the contextual information. Based on the contextual information (e.g., an overlapping or adjacent calendar event is interfering with the user from performing an action corresponding to the therapeutic protocol event), the therapeutic protocol adherence subsystem 123 may determine to perform management of the diabetic condition at 226. For example, the therapeutic protocol adherence subsystem 123 may manage the diabetic condition by adjusting a timer associated with the diabetes-related notification, via one or more of the processes described herein.

The therapeutic protocol adherence subsystem 123 may avoid performing management of the diabetic condition at 220 when the user provides a response to the diabetes-related notification that corresponds to the parameters of the therapeutic protocol event. For example, the therapeutic protocol adherence subsystem 123 may determine that the user administered an injection of insulin at the scheduled time and scheduled dose that corresponds to the parameters of the dosing event. In another example, the therapeutic protocol adherence subsystem 123 may determine that the user measured the fasting blood glucose levels at the scheduled time and/or determine that the measurement values/ranges correspond to the parameters of the measurement event.

For the cases in which the therapeutic protocol adherence subsystem 123 determines to avoid performing management of the diabetic condition, the therapeutic protocol adherence subsystem 123 determines whether a therapeutic target is achieved at 222. For example, the mobile device 104 determines whether the fasting blood glucose level is within a target range (e.g., 80-130 mg/dL). The target range indicates, for example, a glycemic target associated with a proper medication/therapeutic level for treating the medical condition of the user. In another example, the mobile device 104 determines whether the scheduled dose of insulin is the optimal dose of insulin (e.g., a targeted dose of insulin for a particular patient).

For the cases in which the mobile device 104 determines that the therapeutic target is not achieved, the mobile device 104 continues the therapeutic protocol (e.g., basal insulin titration) by determining a subsequent event of a therapeutic protocol at 204. For the cases in which the mobile device 104 determines that the therapeutic target is achieved, the mobile device 104 provides a treatment notification at 224. For example, the mobile device 104 determines that the fasting blood glucose level falls within a target range (e.g., 80-130 mg/dL) for an associated dose of insulin. Having determined the optimal dose of insulin for treating the diabetic condition of the user, the mobile device 104 may display the treatment notification, via a GUI of the associated therapeutic protocol adherence subsystem 123. The treatment notification may include further instructions for the user to follow to treat the diabetic condition. For instance, the treatment notification may indicate that the user should maintain administering the optimal dose of insulin at predetermined time periods.

In one or more cases, the therapeutic protocol adherence subsystem 123 may be configured to learn from the effectiveness of the diabetes-relation notifications, via machine learning algorithms. For example, the therapeutic protocol adherence subsystem 123 may provide a notification to the user requesting the user's input as to whether the user was satisfied with the recent diabetes-related notification. The user, for example, may provide input by selecting “Yes” or “No” as to whether the user was satisfied with the recent diabetes-related notification and/or selecting from a list of reasons related to the user's satisfaction with the diabetes-related notification. The therapeutic protocol adherence subsystem 123 may provide the user's response(s) to the machine learning algorithms to detect the user's individual diabetes-related notification preferences and better tailor the triggering mechanisms. In one or more cases, the determined preferences may be made transparent to the user, for example, by providing a table of diabetes-related notifications and associated triggering mechanisms and triggering details. For example, a diabetes-related notification may be associated with a smart combination of triggering mechanisms (e.g., determining that a user is watching a smart television based on the smart television being powered in an on-state) that are characterized with “auto-detected conditions,” for example, a condition in which diabetes-related notifications are not provided to the user when watching television.

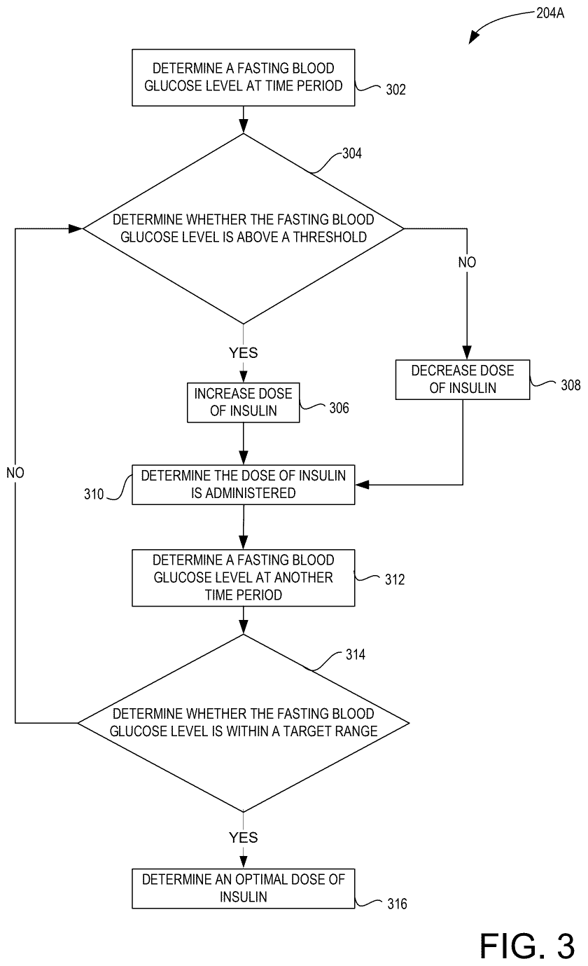

Though the processes 200, 250 are provided for managing adherence to various therapeutic protocols, specific examples are provided herein for managing adherence to basal insulin titration protocols. For example, FIG. 3 is a diagram illustrating an example process 204A for adherence to a therapeutic protocol for titrating basal insulin. One or more portions of the process 204A may be performed by one or more computing devices. For example, the one or more portions of the process 204A may be performed by one or more mobile devices, such as mobile device 104 shown in FIG. 1, and/or one or more remote computing devices, such as remote computing device 122 shown in FIG. 1. One or more portions of the process 204A may be stored in memory as computer-readable or machine-readable instructions that may be executed by a processor of the one or more computing devices. One or more portions of the process 204A may be performed by one or more subsystems operating on one or more computing devices. For example, one or more portions of the process 204A may be performed by one or more devices operating a therapeutic protocol adherence subsystem, such as the therapeutic protocol adherence subsystem 123 shown in FIG. 1. Though portions of the process 204A may be described herein as being performed by a therapeutic protocol adherence subsystem operating on a particular computing device, the process 204A may be performed by another computing device or distributed across multiple computing devices, such as one or more mobile devices and/or remote computing devices.

As shown in FIG. 3, the therapeutic protocol adherence subsystem 123 may determine a fasting blood glucose level at a time period (e.g., an event of the therapeutic protocol) at 302. For example, the therapeutic protocol adherence subsystem 123 may determine the fasting blood glucose level by receiving blood glucose data from one or more of the CGM 102, FGM 103, and/or BGM 106, as described herein. The received blood glucose level corresponds to a sample of blood that was tested during a time period, such as in the morning (e.g., Monday morning) and before the user eats, thereby providing the therapeutic protocol adherence subsystem 123 with the fasting blood glucose level (e.g., the fasting blood glucose level corresponding to Monday morning). The therapeutic protocol adherence subsystem 123 determines at 304 whether the fasting blood glucose level is below a threshold value (e.g., 130 mg/dL). It is noted that the process 204A illustrates obtaining a fasting blood glucose level and determining whether the fasting blood glucose level is above the threshold value. Multiple consecutive fasting blood glucose levels may be obtained, and the protocol adherence subsystem 123 may determine whether an average of the multiple consecutive fasting blood glucose levels are above the threshold value. For example, basal insulin titrations may take an average of multiple consecutive fasting blood glucose values over a period of days to determine if titration is needed. Some basal insulin titration algorithms also include pre/post-meal time blood glucose measurements to determine if meal insulin is required or additional adjustment to basal insulin is needed.

For the cases in which the therapeutic protocol adherence subsystem 123 determines that the fasting blood glucose level is above a threshold value, the therapeutic protocol adherence subsystem 123 increases the insulin dose at 306. For example, the therapeutic protocol adherence subsystem 123 increases the basal insulin dose from the starting therapeutic dose. For instance, the therapeutic protocol adherence subsystem 123 may increase an insulin injection, such as a Lantus®, by two units every three days until an individualized target fasting plasma glucose (FPG) is achieved. In another instance, the therapeutic protocol adherence subsystem 123 may increase the insulin injection, such as Lantus®, by one unit every day until the FPG is less than or equal to 100 mg/dL or until an individualized target FPG is achieved. In yet another instance, the therapeutic protocol adherence subsystem 123 may increase an insulin injection, such as Levemir®, based on an assessment of a three-day FPG mean. In yet another instance, the therapeutic protocol adherence subsystem 123 may increase an insulin injection, such as Levemir®, by three units if the FPG is greater than an upper limit of the FPG range. For example, if the target FPG range is between 70-90 mg/dL, the therapeutic protocol adherence subsystem 123 may increase the insulin injection if the FPG range is greater than 90 mg/dL. In some cases, the increased amount of insulin may be associated with the measured fasting blood glucose level. For the cases in which the therapeutic protocol adherence subsystem 123 determines that the fasting blood glucose level is below a threshold value, the therapeutic protocol adherence subsystem 123 decreases the insulin dose at 308. For example, the therapeutic protocol adherence subsystem 123 may decrease an insulin injection, such as Levemir®, by three units if the FPG is less than a lower limit of the FPG range. For example, if the target FPG range is between 70-90 mg/dL, the therapeutic protocol adherence subsystem 123 may decrease the insulin injection if the FPG range is less than 70 mg/dL. In some cases, the decreased amount of insulin may be associated with the measured fasting blood glucose level.

Having determined the increase or decrease in the amount of insulin to be administered, the therapeutic protocol adherence subsystem 123 determines that the insulin is administered to the user (e.g., another event of the therapeutic protocol) at 310. In some cases, the therapeutic protocol adherence subsystem 123 determines that the insulin is administered to the user by receiving an input into the therapeutic protocol adherence subsystem 123, via the GUI displayed on the mobile device 104, indicating that the user 100 received the insulin injection. In some cases, the input may indicate that the user 100 received the insulin injection. In some cases, the input may correspond to additional contextual information indicating that the user received the insulin injection, the corresponding amount of insulin, and/or when the user received the injection (e.g., Monday evening at 9:00 PM).

The therapeutic protocol adherence subsystem 123 may determine a fasting blood glucose level at another time period (e.g., another event of the therapeutic protocol) at 312. The therapeutic protocol adherence subsystem 123 may determine the fasting blood glucose level as described herein. The received blood glucose level corresponds to a sample of blood that was tested during another time period, such as in the morning on another day (e.g., Tuesday morning) and before the user eats, thereby providing the therapeutic protocol adherence subsystem 123 with the fasting blood glucose level (e.g., the fasting blood glucose level corresponding to Tuesday morning). The therapeutic protocol adherence subsystem 123 determines at 314 whether the fasting blood glucose level is within a target range (e.g., 80-130 mg/dL). The target range indicates, for example, a glycemic target associated with a proper medication/therapeutic level for treating the medical condition of the user.

For the cases in which the therapeutic protocol adherence subsystem 123 determines that the fasting blood glucose level is not within a target range, the therapeutic protocol adherence subsystem 123 determines at 304 whether the fasting blood glucose level is above a threshold value and repeats the process as described herein. It is noted that the process 204A illustrates determining whether the fasting blood glucose level is above the threshold value for the cases in which the therapeutic protocol adherence subsystem 123 determines that the fasting blood glucose level is not within a target range. However, for the cases in which the therapeutic protocol adherence subsystem 123 determines that the fasting blood glucose level is not within a target range, it should be understood that multiple consecutive fasting blood glucose levels may be obtained (e.g., an average of consecutive days of fasting blood glucose levels), and the protocol adherence subsystem 123 may determine whether an average of the multiple consecutive fasting blood glucose levels are above the threshold value. For the cases in which the therapeutic protocol adherence subsystem 123 determines that the fasting blood glucose level is within a target range, the therapeutic protocol adherence subsystem 123 determines an optimal dose of insulin at 316 for treating the diabetic condition of the user. The optimal dose of insulin may correspond to the previous amount of insulin administered to the user, in which the user's fasting blood glucose level falls within the target range. If a hypoglycemic event is recorded, dosage may be reduced due to potentially acute the life-threatening nature of hypoglycemia.

It is noted that the process 204A describes an example process for titrating basal insulin. However, it is understood that the process 204A is exemplary only and other titration protocols (e.g., titration protocols for blood thinners, anti-depressants, statins, anticonvulsants, sedatives, and the like) may be implemented to achieve the optimal dose associated with a proper medication/therapeutic level for treating the medical condition of the user.

In some embodiments, the therapeutic protocol adherence subsystem 123 associates an event of the therapeutic protocol 204A with one or more triggering events. In an example, the titration protocol event of determining a fasting blood glucose level during the morning may be associated with multiple triggering events (e.g., detecting a device display triggering event of the mobile device 104 indicating that the display of the mobile device 104 turns on between 7:00 AM and 8:00 AM, and detecting a device movement triggering event indicating that the mobile device 104 moved for a certain period of time). In another example, the titration protocol event of determining that the user received the insulin injection in the evening may be associated with a triggering event (e.g., receiving an input into the therapeutic protocol adherence subsystem 123, via the GUI displayed on the mobile device 104, indicating that the user received the insulin injection). The titration protocol events and associated triggering events may be stored in the datastore(s) 124. The therapeutic protocol adherence subsystem 123 may be configured to reference the associated titration protocol events and triggering events to detect a triggering event as described herein.

In some embodiments, the titration protocol events may be initially associated with default time periods (e.g., 6:00 AM to 8:00 AM) stored as parameters for the corresponding titration protocol event. As such, the therapeutic protocol adherence subsystem 123 may determine whether a triggering event is detected during the default time period associated with the titration protocol event. In some embodiments, the therapeutic protocol adherence subsystem 123 may determine whether a triggering event is detected throughout the course of the associated therapeutic protocol event. For example, if the titration protocol event is to determine a fasting blood glucose level, the therapeutic protocol adherence subsystem 123 may determine whether a triggering event is detected until the therapeutic protocol adherence subsystem 123 determines the fasting blood glucose level as described herein.

In some embodiments, a triggering event includes activities that characterize a circadian rhythm of a user (e.g., user 100). In some examples, a triggering event may be associated with a default time or timeframe. For instance, a triggering event may be associated with a timeframe of 8:00 AM to 10:00 AM. In some examples, a triggering event may correspond to a default timeframe associated with a respective day of the week. For instance, one triggering event may include a timeframe of 8:00 AM to 10:00 AM for a Monday, and another triggering event may include a time of 10:30 AM for a Saturday. In some other examples, a triggering event may correspond to a device display triggering event that indicates a first instance that the screen of the mobile device 104 turns on within a timeframe (e.g., a timeframe of 8:00 AM to 10:00 AM). Such a triggering event may indicate that a user (e.g., user 100) interacts with the mobile device 104 and is thus capable of engaging in a therapeutic protocol event (e.g., measuring a fasting blood glucose level). In some other examples, a triggering event may correspond to a device movement triggering event indicating a first instance that the mobile device 104 moves within a timeframe. For instance, a mobile device 104 may detect a movement via, for example, an integrated accelerometer or gyroscope, and provide an indication that the mobile device 104 changed positions from a resting state to another state. Such a triggering event may indicate that a user interacts with the mobile device 104 and is thus capable of engaging in a therapeutic protocol event.

In some other examples, the trigger event may be associated with a biometric parameter of the user that is tracked via a connected smart device, such as the mobile device 104 or connected smart device 107, which may include but is not limited to a heart rate monitor, a smart watch, a smart bed, and other like smart devices capable of indicating wake or sleep states of the user. In some cases, the biometric parameters may be monitored via the therapeutic protocol adherence subsystem 123, within a lightweight application similar to the therapeutic protocol adherence subsystem 123, or indirectly through other applications which are active in the background of the operating system of the therapeutic protocol adherence subsystem 123. In some examples, biometric parameters (e.g., heart rate) may be used to observe wake-up patterns via measured and sustained increases in the monitored biometric parameter.

In some other examples, the triggering event may be associated with a motion parameter of the user that is tracked via a connected smart device, such as the RF beacon device 126, IOT device 127, and other like devices that may provide an indication of the user's behavior. In an example, the mobile device 104 may utilize one or more RF beacon devices 126 to determine that the mobile device 104 is moved from a bedroom of the user 100 to the kitchen of the user 100, thereby providing an indication that the user 100 is in a wake state. In another example, the mobile device 104 may utilize an IOT device 127, such as a smart thermostat, to determine that the user 100 adjusted the temperature of the smart thermostat. In another example, the mobile device 104 may utilize an IOT device 127, such as a smart refrigerator, coffee machine, and the like, to determine that the user 100 interacted with the IOT device 127 (e.g., removing an item from the smart refrigerator).

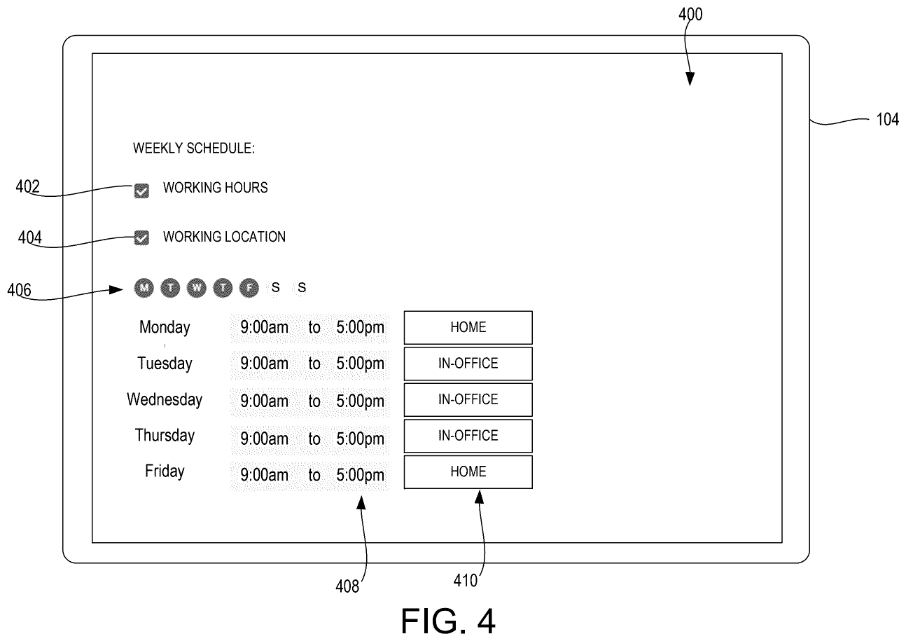

In some other examples, a user may schedule one or more triggering events to indicate a user's daily schedule and/or provide optimal times to execute a therapeutic protocol event, such as measuring blood glucose levels and/or administering a dosage of insulin. For instance, the user may access a scheduling window, via a GUI displayed on the mobile device 104 via the therapeutic protocol adherence subsystem 123. FIG. 4 illustrates an example GUI including a scheduling window 400 that may display scheduling information to schedule a triggering event. In some cases, the scheduling window 400 may include a weekly schedule that allows the user 100 to indicate working hours (e.g., by selecting the working hours icon 402) and/or a working location (e.g., by selecting the working location icon 404). The scheduling window 400 may provide an option for the user to select the days that the user works (e.g., by selecting a day icon 406, such as by selecting the Monday, Tuesday, Wednesday, Thursday, and Friday icons). Further, for the selected working days, the scheduling window 400 icons may be used to customize the day of the user 100. For example, if the working hours icon 402 is selected, the scheduling window 400 may display the corresponding selected working days and a respective timeframe icon 408. The user 100 may provide input into a respective timeframe icon 408 to set working hours (e.g., a time window icon 408 may indicate that working hours for Mondays are 9:00 a.m. to 5:00 p.m.). In another example, if the working location icon 404 is selected, the scheduling window 400 may display the corresponding selected working days and a respective location icon 410. The user 100 may provide input into a respective location icon 410 to indicate where the user 100 is working on a respective day (e.g., a location icon 410 may indicate that the user 100 is working at home on Mondays and is working in the office on Tuesdays). In some embodiments, the therapeutic protocol adherence subsystem 123 may modify the parameters of therapeutic protocol events and/or triggering events for notifications based on the selections in the scheduling window 400. As such, a user's work schedule with a typical day and/or night activity levels (e.g., night shift workers) may be accommodated to manage therapeutic protocols.

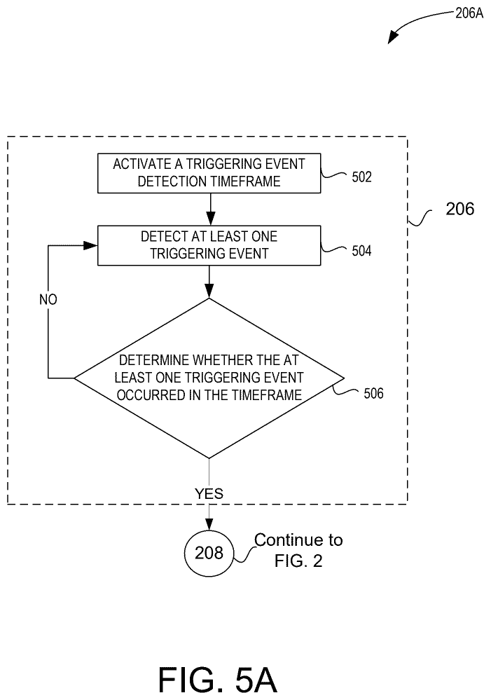

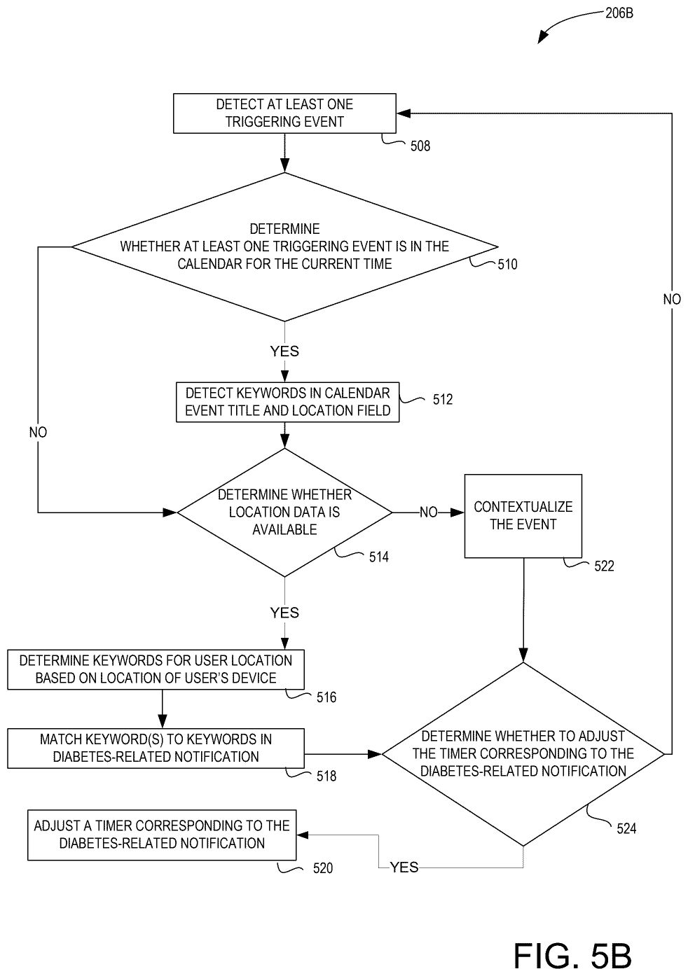

After the timeframe is defined for an event of the therapeutic protocol (e.g., as shown in the scheduling window 400 in FIG. 4), the therapeutic protocol adherence subsystem 123 may determine whether a triggering event for notifying the user 100 is received within the defined timeframe. FIG. 5A shows a flowchart of an example triggering event detection process 206A for detecting triggering events during a define timeframe. One or more portions of the triggering event detection process 206A may be performed by one or more computing devices. For example, the one or more portions of the triggering event detection process 206A may be performed by one or more mobile devices, such as mobile device 104 shown in FIG. 1, and/or one or more remote computing devices, such as remote computing device 122 shown in FIG. 1. One or more portions of the triggering event detection process 206A may be stored in memory as computer-readable or machine-readable instructions that may be executed by a processor of the one or more computing devices. One or more portions of the triggering event detection process 206A may be performed by one or more subsystems operating on one or more computing devices. For example, one or more portions of the triggering event detection process 206A may be performed by one or more devices operating a therapeutic protocol adherence subsystem, such as the therapeutic protocol adherence subsystem 123 shown in FIG. 1. In fact, as shown in FIG. 5A, one or more portions of the triggering event detection process 206A may be performed when the therapeutic protocol adherence subsystem 123 performs the detection of one or more triggering events as illustrated at step 206 of the processes 200, 250 shown in FIGS. 2A and 2B. However, portions of the process 206A, or portions thereof, may be performed independently of the processes 200, 250 shown in FIGS. 2A and 2B. Additionally, though portions of the process 206A may be described herein as being performed by a therapeutic protocol adherence subsystem operating on a particular computing device, the process 206A may be performed by another computing device or distributed across multiple computing devices, such as one or more mobile devices and/or remote computing devices.

As illustrated in the example triggering event detection process 206A of FIG. 5A, a triggering event detection timeframe is activated at 502. The therapeutic protocol adherence subsystem 123 activates the triggering event detection timeframe having determined a therapeutic protocol event, as discussed herein. The triggering event detection timeframe may be associated with one or more corresponding triggering events and the determined therapeutic protocol event. In some embodiments, the therapeutic protocol adherence subsystem 123 detects at 504 at least one triggering event and determines at 506 whether the at least one triggering event occurred in the activated triggering event detection timeframe.

For the cases in which the therapeutic protocol adherence subsystem 123 determines that the at least one triggering event did not occur in the activated triggering event detection timeframe, the therapeutic protocol adherence subsystem 123 continues to monitor for triggering events. In some cases, the therapeutic protocol adherence subsystem 123 may determine that the at least one triggering event did not occur in the activated triggering event detection timeframe if the at least one triggering event did not occur within the activated timeframe. The activated timeframe may be adjusted based on adjustments or tolerance levels, as described herein. As such, the therapeutic protocol adherence subsystem 123 prevents inadvertent or inaccurate diabetes-related notifications from being provided to the therapeutic protocol adherence subsystem 123 when triggering events occur outside of the timeframe designated by the therapeutic protocol. For the cases in which the therapeutic protocol adherence subsystem 123 determines that the at least one triggering event did occur in the activated triggering event detection time period, the therapeutic protocol adherence subsystem 123 continues to initiate a diabetes-related notification (e.g., as indicated at 208 of the processes 200, 250 as illustrated in FIGS. 2A and 2B and/or as further described herein).

The therapeutic protocol adherence subsystem 123 may determine whether a triggering event for notifying the user 100 is received based on location information, calendar information, or a combination of location information and calendar information. FIG. 5B shows a flowchart of an example triggering event detection process 206B for detecting triggering events from a user's calendar and/or a location of the user. One or more portions of the triggering event detection process 206B may be performed by one or more computing devices. For example, the one or more portions of the triggering event detection process 206B may be performed by one or more mobile devices, such as mobile device 104 shown in FIG. 1, and/or one or more remote computing devices, such as remote computing device 122 shown in FIG. 1. One or more portions of the triggering event detection process 206B may be stored in memory as computer-readable or machine-readable instructions that may be executed by a processor of the one or more computing devices. One or more portions of the triggering event detection process 206B may be performed by one or more subsystems operating on one or more computing devices. For example, one or more portions of the triggering event detection process 206B may be performed by one or more devices operating a therapeutic protocol adherence subsystem, such as the therapeutic protocol adherence subsystem 123 shown in FIG. 1. In fact, as shown in FIG. 5B, one or more portions of the triggering event detection process 206B may be performed when the therapeutic protocol adherence subsystem 123 performs the detection of one or more triggering events as illustrated at step 206 of the processes 200, 250 shown in FIGS. 2A and 2B. However, the process 206B, or portions thereof, may be performed independently of the processes 200, 250 shown in FIGS. 2A and 2B. Additionally, though portions of the process 206B may be described herein as being performed by a therapeutic protocol adherence subsystem operating on a particular computing device, the process 206B may be performed by another computing device or distributed across multiple computing devices, such as one or more mobile devices and/or remote computing devices.

The therapeutic protocol adherence subsystem 123 detects at least one triggering event, as described herein. In response to detecting the triggering event, the therapeutic protocol adherence subsystem 123 determines whether the user has previously given permission to access the user's personal calendar. If the user has given permission to access a personal calendar of the user, the therapeutic protocol adherence subsystem 123 determines whether there is a triggering event in the calendar for the current time or within a predefined period of time at 510. For example, upon detection of a triggering event, the therapeutic protocol adherence subsystem 123 accesses the user's calendar and determines that there is calendar information, for example, but not limited to, an event for the current time or within a predefined period of time. The calendar is accessed by the therapeutic protocol adherence subsystem 123 locally, from a remote datastore, from a remote computing device, or from a calendar service.

It is noted that the examples provided herein discuss accessing one calendar to determine whether there is a triggering event. However, it is understood that the therapeutic protocol adherence subsystem 123 may access multiple calendars to determine whether there is a triggering event for the current time or within a predefined period of time. Further, in some cases, upon setup of the therapeutic protocol adherence subsystem 123, the user may choose to select ‘personalized reminders’ and allow referencing of their calendar to adjust notification times to coincide with times where adherence to the notifications is more likely to occur. The therapeutic protocol adherence subsystem 123 may be configured to read and interpret the calendar information via a semantic referential database or other architecture that associates keywords or dates/times in calendar events with functional logic. In some cases, the therapeutic protocol adherence subsystem 123 may be configured to extract more advanced, information from semantic calendar entries (e.g., a working time may be determined based on meetings with other people on invite list or appointment name/description indicating a certain work task).

If there is no event in the calendar at the current time or within a predefined period of time, or if the user has not given permission to access the calendar, the therapeutic protocol adherence subsystem 123 proceeds to 514. If there is calendar information, for example, an event in the calendar at the current time or within a predefined period of time, the therapeutic protocol adherence subsystem 123 detects one or more keywords in the title field of the event or in the location field of the event at 512. For example, the event title is “Breakfast with John” and the event location is “Cafe”. The therapeutic protocol adherence subsystem 123 detects the keyword “breakfast” in the title field or the keyword “Cafe” in the location field. If the location is a street address, the user device attempts to match the street address to a known entity (e.g., a business, residence, or other entity) at that street address and detect keywords in the name of the known entity. The name of the known entity is a friendly name. For example, if the location is the street address “329 Sanderson Street”, the therapeutic protocol adherence subsystem 123 determines that the known entity at that street address is the “Cafe” and uses the keyword “Cafe” for the location field. In another example, the event title is “take cholesterol medication” but does not include a keyword in the location field. The therapeutic protocol adherence subsystem 123 detects the keyword “medication”.

At 514, the therapeutic protocol adherence subsystem 123 determines whether location information (e.g., GPS data) is available to the therapeutic protocol adherence subsystem 123. Location information may not be available to the therapeutic protocol adherence subsystem 123 if, for example, the therapeutic protocol adherence subsystem 123 is not in range of a GPS signal, or the user has not given permission to use location-based services or access their location.

If the location information is unavailable, the therapeutic protocol adherence subsystem 123 proceeds to 522 and contextualizes the triggering event. For example, if the therapeutic protocol adherence subsystem 123 detects the keyword “medication” that occurs at 8:00 PM for the event, the therapeutic protocol adherence subsystem 123 determines that the user is scheduled to take cholesterol medication at 8:00 PM. Based on the contextualized event, the therapeutic protocol adherence subsystem 123 determines whether to adjust a timer corresponding to the diabetes-related notification at 524. In some embodiments, the therapeutic protocol adherence subsystem 123 adjusts at 520 a timer corresponding to the diabetes-related notification. A diabetes-related notification may correspond to a notification to perform an action associated with a therapeutic protocol event.

In some cases, the therapeutic protocol adherence subsystem 123 adjusts the timer of the diabetes-related notification based on the contextualized event being similar or associated with the activity associated with the diabetes-related notification (e.g., both events are associated with administering or managing medication; or the activities of both events are tangentially related, such as administering insulin and performing diagnostic measurements (such as blood pressure, weight, blood glucose, and the like), recording or reviewing diet entries, exercising, or performing other regimented health and wellness related activities that may be a part of a plan from a healthcare provider). For example, diabetes-related notification for providing an indication to the user to administer an insulin dose may be set by default to occur at 9:00 PM. Based on the therapeutic protocol adherence subsystem 123 determining that the user is scheduled to take cholesterol medication at 8:00 PM and scheduled to administer insulin at 9:00 PM, the therapeutic protocol adherence subsystem 123 may adjust the diabetes-related notification such that the notification is provided to coincide with cholesterol medication event. That is, the therapeutic protocol adherence subsystem 123 may reschedule the administering of insulin notification to be provided with a notification to take cholesterol medication and/or to schedule the diabetes-related notification such that the administering of insulin now occurs at 8:00 PM. Timers of the diabetes-related notifications may be adjusted to coincide with or near a time period of other previously scheduled calendar events as a form of convenience for the user and to minimize the cognitive load of having to response to multiple notifications/events throughout the day.

In another example, a timer of a diabetes-related notification (e.g., measure fasting blood glucose levels) may be scheduled to occur in the morning, for example, at 7:00 AM, by default. If the calendar includes, for example, an event title “Breakfast with Paul” scheduled to occur at 6:30 AM, the therapeutic protocol adherence subsystem 123 detects the keyword “breakfast” in the title field and “6:30 AM” in the event time field, and determines that the user is scheduled to have breakfast at 6:30 AM (i.e., prior to the time at which the diabetes-related notification of measuring fasting blood glucose levels is scheduled to occur). Based on the contextualized event, the therapeutic protocol adherence subsystem 123 adjusts the timer of the diabetes-related notification to occur prior to the scheduled breakfast. In some cases, the timer of the diabetes-related notification may be adjusted to occur as the earliest scheduled event in the user's calendar. In some cases, the timer of the diabetes-related notification may be adjusted to occur at a predetermined period of time before the contextualized event. For example, the therapeutic protocol adherence subsystem 123 may adjust the timer of diabetes-related notification of measuring fasting blood glucose levels to occur at 6:00 AM. In some cases, the therapeutic protocol adherence subsystem 123 may be configured to provide a prompt, via the GUI of the therapeutic protocol adherence subsystem 123, for the user to confirm such changes to the timer of the diabetes-related notification. In other cases, the therapeutic protocol adherence subsystem 123 may be configured to automatically change the timer of the diabetes-related notification.

In some cases, the therapeutic protocol adherence subsystem 123 determines that the contextualized event is not similar or associated with the activity associated with the diabetes-related notification. For example, the therapeutic protocol adherence subsystem 123 may determine that adjusting the timer of the diabetes-related notification to coincide with the occurrence of the contextualized event would not be convenient for the user. The therapeutic protocol adherence subsystem 123 may determine that an open time slot within the calendar would be more convenient for the user to perform the activity associated with the diabetes-related notification. In such cases, the therapeutic protocol adherence subsystem 123 may adjust the timer associated with the diabetes-related notification to occur in the open time slot.

In one or more cases, the therapeutic protocol adherence subsystem 123 may be configured to extend a timer associated with a diabetes-related notification or an acceptable activity window for logging information related to the diabetes-related notification based on a day of the week (e.g., a Saturday or Sunday) and/or if calendar event information suggests that the user is traveling and may be experiencing jet lag.

In some cases, if the therapeutic protocol adherence subsystem 123 determines that no calendar event information is available, the therapeutic protocol adherence subsystem 123, via the therapeutic protocol adherence subsystem 123, may determine and/or set timers associated with diabetes-related notification based on sleep and/or wake settings within the operating system of the therapeutic protocol adherence subsystem 123. In such cases, the therapeutic protocol adherence subsystem 123 may be configured to set and/or adjust timers to occur at the sleep and/or wake times or at a time period after the sleep and/or wake times.

If the location information is available, the therapeutic protocol adherence subsystem 123 determines one or more keywords for the user location based on the location information at 516. For example, the data indicates the location of the therapeutic protocol adherence subsystem 123 as a set of latitude and longitude coordinates. The therapeutic protocol adherence subsystem 123 uses a location information base to match the coordinates to an address. In some cases, the therapeutic protocol adherence subsystem 123 may be configured to read and interpret the location information via a referential database or other architecture that associates keywords with functional logic. In some cases, the therapeutic protocol adherence subsystem 123 may use standard calls to a location services API to read the location information. The therapeutic protocol adherence subsystem 123 uses the street address to determine one or more keywords for a known entity at that address. For example, the therapeutic protocol adherence subsystem 123 obtains its latitude and longitude coordinates from GPS data, and determines that the street address matching those coordinates is “639 Sanderson Street.” If the known entity found at the street address has one or more metadata fields that are useful to categorizing the type of event or location at that known entity in its GPS database information, the therapeutic protocol adherence subsystem 123 detects keywords in the metadata fields. For example, known entity “Brown's Gym” at street address “639 Sanderson Street” may have one or more metadata fields indicating that the address corresponds to the local gym, “Brown's Gym”, and the user device detects the keyword “Gym” in the metadata fields.

It is noted that upon setup of the therapeutic protocol adherence subsystem 123, a user may choose to select personalized notifications and allow referencing of their location to adjust timers of diabetes-related notifications to coincide with times at which adherence to the diabetes-related notifications are more likely to occur.

At 518, the therapeutic protocol adherence subsystem 123 matches the keywords from the calendar and/or the geographic location information to keywords associated with pre-determined diabetes-related notifications. The diabetes-related notifications are stored in a database on the therapeutic protocol adherence subsystem 123, an external device, or an external datastore, such as datastore(s) 124. The diabetes-related notifications are associated with one or more keywords. For example, if the therapeutic protocol adherence subsystem 123 detects the keywords “workout” and/or “gym” that occurs at 7:00 AM for the event and detects the keyword “Gym” as a location of the event, the therapeutic protocol adherence subsystem 123 determines that the user is scheduled to exercise at Brown's Gym at 7:00 AM. Based on the contextualized event, the therapeutic protocol adherence subsystem 123 determines whether to adjust a timer corresponding to the diabetes-related notification at 524.

In some embodiments, the therapeutic protocol adherence subsystem 123 adjusts at 520 a timer corresponding to the diabetes-related notification. For example, a diabetes-related notification (e.g., measuring a fasting blood glucose level) may be set by default to occur in the morning (e.g., 7:15 AM). Based on the contextualized event, the therapeutic protocol adherence subsystem 123 determines that the user would be at the gym when the diabetes-related notification is set to occur, and may determine that the user would be unavailable to take a fasting blood glucose measurement. For instance, the user may be exercising or the user does not have a BGM or CGM while at the gym. In some cases, the therapeutic protocol adherence subsystem 123 adjusts the timer to occur prior to the scheduled event. In other cases, the therapeutic protocol adherence subsystem 123 is configured to display the diabetes-related notification as scheduled. In other cases, the therapeutic protocol adherence subsystem 123 is configured to provide the diabetes-related notification to another connected device capable of displaying the diabetes-related notification to the user. For example, the other connected device may be a smartwatch or another device within the vicinity of the user. By displaying the diabetes-related notification on other devices, the chances of user awareness and adherence to the diabetes-related notification are increased.

In another example, the therapeutic protocol adherence subsystem 123 may determine that it is unsafe for the user to perform the action related to the diabetes-related notification. For example, the therapeutic protocol adherence subsystem 123 may determine that the user is driving a vehicle when a diabetes-related notification is about to occur. In some cases, the therapeutic protocol adherence subsystem 123 determines that the user is driving based on the location information. In other cases, the therapeutic protocol adherence subsystem 123 determines that the user is driving based on applications (e.g., navigation-type applications) that are executed and being used on the therapeutic protocol adherence subsystem 123. In yet other cases, the therapeutic protocol adherence subsystem 123 may infer that the user is driving based on calendar event information. For the cases in which the therapeutic protocol adherence subsystem 123 determines that it is unsafe for the user to perform the action related to the diabetes-related notification, the therapeutic protocol adherence subsystem 123 may delay or suspend providing the diabetes-related notification to the user. For instance, based on the location information, the therapeutic protocol adherence subsystem 123 may suspend the diabetes-related notification until the therapeutic protocol adherence subsystem 123 determines that the user is no longer driving.

In another example, the therapeutic protocol adherence subsystem 123 modifies a delivery method and/or delivery destination of the diabetes-related notification based on the contextualized event and/or an importance of the diabetes-related notification. In some cases, the therapeutic protocol adherence subsystem 123 may modify the delivery method and/or delivery destination of the diabetes-related notification by providing the diabetes-related notification to a device that is different from the default destination (e.g., a mobile device, such as mobile device 104). For example, the therapeutic protocol adherence subsystem 123 may modify the delivery destination of the diabetes-related notification from being the mobile device 104 to another connected device (e.g., a smartwatch or other smart device within the vicinity of the user) capable of indicating the diabetes-related notification to the user. In some cases, the therapeutic protocol adherence subsystem 123 may modify the delivery method and/or delivery destination of the diabetes-related notification by providing the diabetes-related notification to the default device and a device (e.g., a smartwatch or other smart device within the vicinity of the user) that is different from the default destination.

In some cases, the therapeutic protocol adherence subsystem 123 may modify the delivery method and/or delivery destination of the diabetes-related notification by providing the diabetes-related notification to the user, via an alternative application and/or a manner (e.g., alerts, announcements, notifications, and the like) in which the mobile device provides the diabetes-related notification. For example, the therapeutic protocol adherence subsystem 123 may modify the delivery destination of the diabetes-related notification from being displayed via a default application to being displayed on an alternative application, such as, but not limited to, the user's email application. For instance, the therapeutic protocol adherence subsystem 123 may provide the diabetes-related notification as an email to the user's email address. In another example, the therapeutic protocol adherence subsystem 123 may provide the diabetes-related notification via one or more sensory channels, such as a visual alert, a haptic alert, an auditory alert, or an alert via another sensory channel, for example. The therapeutic protocol adherence subsystem 123 may modify the default manner of providing the diabetes-related notification from providing an indication of the diabetes-related notification via a haptic vibration to providing the indication via haptic vibration and a sound. In another example, the therapeutic protocol adherence subsystem 123 may modify the delivery method and/or delivery destination of the diabetes-related notification by providing the diabetes-related notification to another connected device (e.g., a smartwatch or other smart device within the vicinity of the user) and by modifying the manner in which the diabetes-related notification is indicated. For instance, the therapeutic protocol adherence subsystem 123 may have a default configuration to provide the diabetes-related notification to a user's mobile device and notify the user of the diabetes-related notification via a vibration notification. The therapeutic protocol adherence subsystem 123 may modify the default configuration such that the diabetes-related notification is now provided to a smart speaker that indicates the diabetes-related notification via a voice notification. The modification may come in response to a failure of a user to acknowledge the way the diabetes-related notification is being provided within a defined period of time.

In some cases, the therapeutic protocol adherence subsystem 123 may modify the delivery method and/or delivery destination of the diabetes-related notification based on the contextualized event. The contextualized event may be determined from a location, a calendar event, or other information accessible to the therapeutic protocol adherence subsystem 123. For example, based on the contextualized event, such as calendar event information and/or location information, the therapeutic protocol adherence subsystem 123 may determine that the user is at the gym when the diabetes-related notification is set to occur, and determine that the user would be unavailable to take a fasting blood glucose measurement. As such, the therapeutic protocol adherence subsystem 123 may provide the diabetes-related notification to a connected device (e.g., a smartwatch or other smart device within the vicinity of the user) capable of indicating (e.g., displaying) the diabetes-related notification to the user. In another example, based on a contextualized event, the therapeutic protocol adherence subsystem 123 may determine that the user is in a location in which the user has limited access to the user's mobile device (e.g., work location, gym, theater, or other location identified by contextualized event). As such, the therapeutic protocol adherence subsystem 123 may provide the diabetes-related notification to a connected device (e.g., a smart speaker capable of playing a voice notification of the diabetes-related notification) and/or an alternative application (e.g., providing the diabetes-related notification as an email to a user's email address). The connected device may be determined to be at the location of the user prior to providing the notification.

In some cases, the therapeutic protocol adherence subsystem 123 may modify the delivery method and/or delivery destination of the diabetes-related notification based on the importance of the notification (e.g., a critical notification may be a notification that provides an action to prevent a user from becoming hypoglycemic or hyperglycemic). For example, based on a notification indicating that the user administer a basal insulin dose, the therapeutic protocol adherence subsystem 123 may provide the diabetes-related notification to the default device, a connected device (e.g., a smart speaker) and/or an alternative application (e.g., an email application associated with a user's email address).

In another example, a diabetes-related notification (e.g., administer a basal insulin dose) may be set by default to occur at a certain time period (e.g., in the evening at 9:00 PM). In some cases, the therapeutic protocol adherence subsystem 123 may be configured to access the location information of the therapeutic protocol adherence subsystem 123 at regular intervals of time to determine the location of the user. For instance, the therapeutic protocol adherence subsystem 123 may determine that the user is located at or near a movie theater. In some cases, if there is no calendar event information, the therapeutic protocol adherence subsystem 123 may contextualize the event based on the location information alone. For instance, based on the location information indicating that the therapeutic protocol adherence subsystem 123 is at or near a movie theater, the therapeutic protocol adherence subsystem 123 may determine that the user is unavailable and may reschedule the diabetes-related notification to occur in a future time period. For example, the therapeutic protocol adherence subsystem 123 may reschedule the diabetes-related notification to occur in two hours. In another instance, based on the location information, the therapeutic protocol adherence subsystem 123 may delay the diabetes-related notification until the location corresponding to the location information changes and/or indicates that the user is at home or a default location. In one or more cases, the therapeutic protocol adherence subsystem 123 may provide a notification to the user before changing the timer of the diabetes-related notification, such that the user may determine whether to change the timer of the diabetes-related notification. In other cases, the therapeutic protocol adherence subsystem 123 automatically adjusts the timer of the diabetes-related notification with or without providing a notification and confirmation to the user.

It is noted that a diabetes-related notification may be configured with one or more default triggering mechanisms. The diabetes-related notification may be associated with default triggering mechanisms that are conducive to certain times of day. For example, a diabetes-related notification may be associated with activity-based triggers (e.g., detecting movement of a mobile device) and/or biometric-based triggers for time periods in which calendar or location information (e.g., GPS data) is unlikely to provide valuable context, if any, such as in the morning hours. In another example, a diabetes-related notification may be associated with calendar and location based triggers for time periods in which a user is likely awake.

In one or more cases, a user may interact with the therapeutic protocol adherence subsystem 123 to configure triggering mechanisms for various diabetes-related notifications and/or associated timers throughout the day. A triggering mechanism may be based on a fixed time, calendar-based, activity-based, or a combination of the aforementioned. For example, a triggering event may be scheduled to occur at a fixed time per day and/or may include exceptions to the fixed triggering event. In another example, a triggering event may be scheduled in free time slots of a calendar or may be scheduled during a specified block of time (e.g., a block of time associated with measuring blood glucose levels). In an example, a morning diabetes-related notification (e.g., measuring fasting blood glucose levels) may be associated with an activity-based triggering mechanism that occurs with a first interaction with a user's mobile device between 6:00 AM and 8:00 AM. In another example, an evening diabetes-related notification (e.g., administering an insulin dose) may be associated with a calendar-based or GPS-based triggering mechanism that occurs during any of the free time slots after 7:00 PM. In another example, an afternoon diabetes-related notification (e.g., administering medication) may be associated with a fixed time or calendar-based triggering mechanism based on the presence of other medication notifications that occurs at a predetermined time (e.g., Monday at 4:00 PM, Tuesday at 5:00 PM, Wednesday at 4:00 PM, Thursday at 5:00 PM, Friday at 4:30 PM, Saturday at 6:00 PM, Sunday at 6:00 PM, and an exception occurring at 5:30 PM on May 13, 2022). In another example, an activity diabetes-related notification (e.g., performing an exercise or workout) may be associated with a smart combination of triggering mechanisms, such as calendar-based triggering mechanisms and biometric triggering mechanisms, that occur, for example, two to three times per week based on one or more conditions, such as, scheduling the triggering mechanisms to occur in one or more free time slots after 4:00 PM on Monday, Tuesday, and Thursday, or scheduling the triggering mechanisms to occur immediately when an average heart rate is less than, for example, eighty beats per minute for more than two consecutive days.

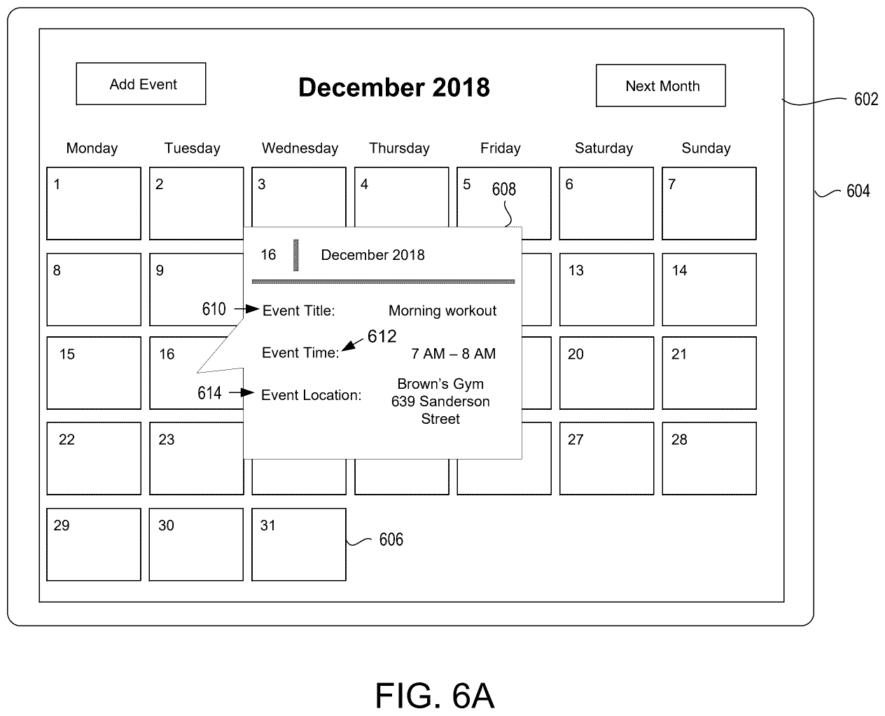

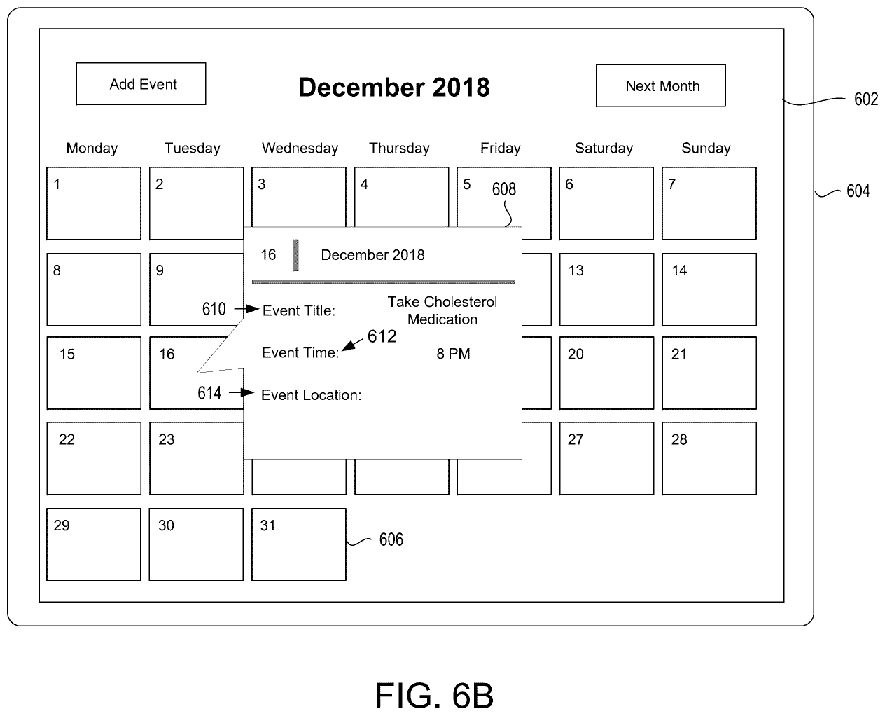

FIGS. 6A and 6B illustrate an example graphical user interface (GUI) 602 that displays calendar information used to determine the location or an activity of a user. For example, the GUI 602 is displayed, via the therapeutic protocol adherence subsystem 123, on a mobile device 604 (e.g., mobile device 104). Mobile device 604 is any user device with a display that is capable of accessing calendar information. For example, mobile device 604 is a smartphone, a wearable device, a laptop, a tablet, a BGM, a CGM controller, or the like.

The GUI 602 displays a calendar 606. The calendar 606 is shown including hours, days, months, and the like. The GUI 602 displays a popup 608 that displays information about a specific calendar event. The information includes the event title 610, the event time 612, or the event location 614. The therapeutic protocol adherence subsystem 123 uses the event time 612 to determine whether there is a current event at the time a triggering event is detected and whether the event is occurring in a predefined period of time. The therapeutic protocol adherence subsystem 123 uses the event title 610 or the event location 614 to determine keywords for the event. The therapeutic protocol adherence subsystem 123 matches the keywords to one or more diabetes-related notifications as described herein.

For example, as illustrated in FIG. 6A, the therapeutic protocol adherence subsystem 123 detects a triggering event at 7:00 AM on December 16th. The mobile device 604 accesses the user's calendar 606 and determines that the user has one event on the calendar 606 for December 16th. The therapeutic protocol adherence subsystem 123 determines from the event time 612 that the event is currently occurring or is occurring in a predefined period of time. The therapeutic protocol adherence subsystem 123 detects the keyword “workout” in the event title 610 and the keyword “gym” in the event location 614. The therapeutic protocol adherence subsystem 123 matches the keywords “workout” or “gym” to the diabetes-related notification and adjusts a timer corresponding to the diabetes-related notification, as described herein.

In another example, as illustrated in FIG. 6B, the therapeutic protocol adherence subsystem 123 detects a triggering event at 8:00 PM on December 16th. The therapeutic protocol adherence subsystem 123 accesses the user's calendar 606 and determines that the user has another event on the calendar 606 for December 16th. The therapeutic protocol adherence subsystem 123 determines from the event time 612 that the event is currently occurring or is occurring in a predefined period of time. The therapeutic protocol adherence subsystem 123 detects the keyword “medication” in the event title 610. The therapeutic protocol adherence subsystem 123 matches the keyword “medication” to the diabetes-related notification and adjusts a timer corresponding to the diabetes-related notification, as described herein.

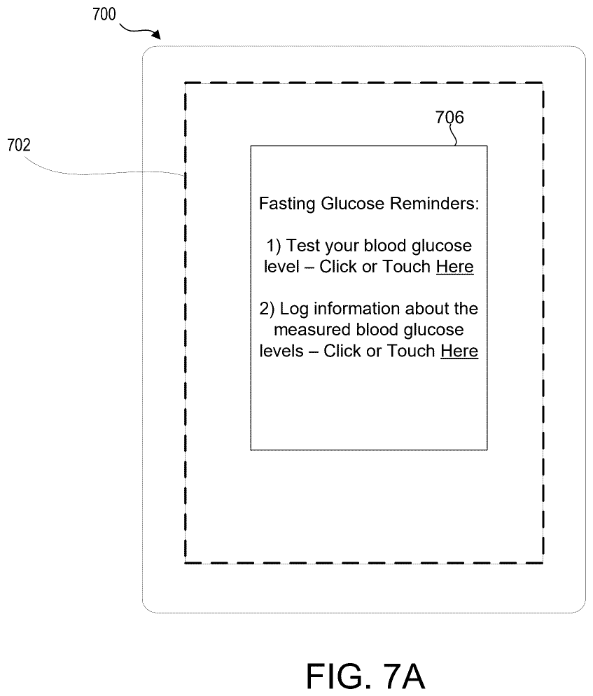

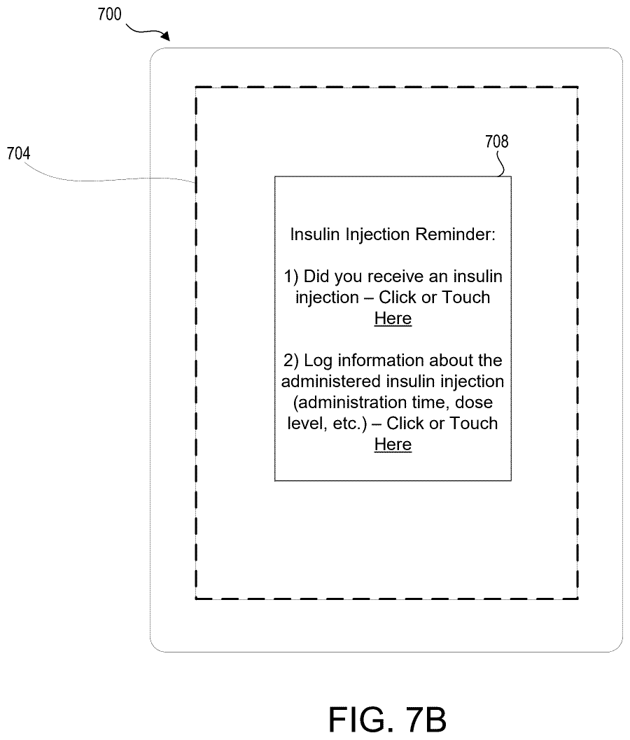

FIGS. 7A and 7B illustrate example GUIs 702, 704 that include diabetes-related notifications 706, 708 that are provided, via therapeutic protocol adherence subsystem 123, on a user device 700 (e.g., mobile device 104). As illustrated in FIGS. 7A and 7B, diabetes-related notifications 706, 708 are provided via a user device 700. The diabetes-related notifications 706, 708 are provided via respective GUIs 702, 704 that are generated locally at the user device 700, or at a remote computing device and displayed via a local application (e.g., web browser or application giving access to therapeutic protocol adherence subsystem 123).

As described herein, the diabetes-related notifications 706, 708 are a form of management of the diabetic condition and include a prompt for the user (e.g., user 100) to perform an action associated with the diabetes-related notification. Based on the detection of a triggering event, an associated diabetes-related notification is provided to the user via a respective GUI. The diabetes-related notification is provided based on the associated timer, as described herein. In an example, the diabetes-related notification, such as diabetes-related notification 706, is provided to the user via the GUI 702 at the scheduled time to measure fasting blood glucose levels. In another example, the diabetes-related notification, such as diabetes-related notification 708, is provided to the user via the GUI 704 at the scheduled time to inject a dose of insulin.

The manner in which the diabetes-related notifications 706, 708 are provided to the user depends on the detected triggering event. Similarly, the form of management of the diabetic condition that the diabetes-related notifications 706, 708 indicate that the user should perform depends on the detected triggering events. The diabetes-related notifications 706, 708 includes buttons or links for the user to easily and quickly perform the indicated form of management of the diabetic condition. For example, the user may input a response to the diabetes-related notification 708 as to whether the user received an insulin injection. In another example, the user may input information associated with the diabetes-related notification 708, such as the administration time of the insulin injection, the dosing amount of the administered insulin, and like information. Accordingly, the buttons or links associated with the diabetes-related notifications 706, 708 increase the likelihood that the user performs management of the diabetic condition in a timely manner and logs relevant information for completing the therapeutic protocol.

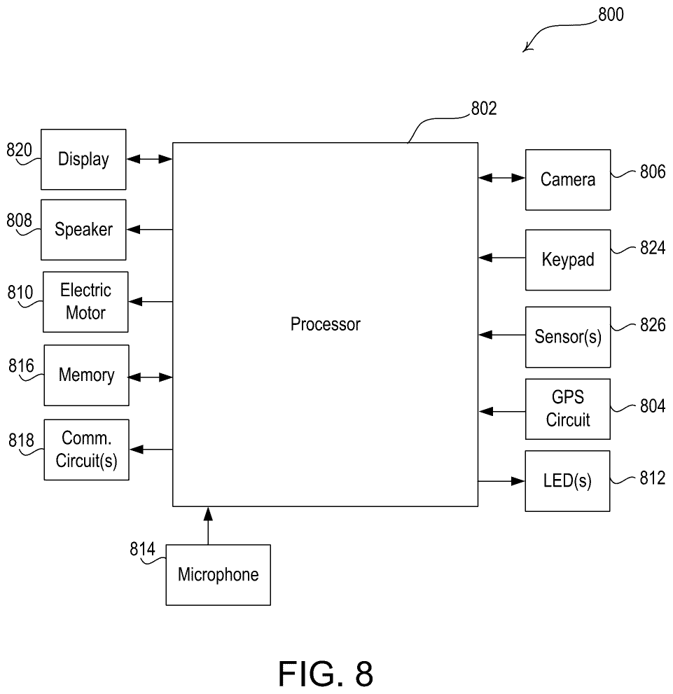

FIG. 8 is a block diagram of an example computing device 800. The computing device is a mobile computing device, such as a tablet, a cellular phone, a wearable device, a CGM controller device, or another computing device, for example. As shown in FIG. 8, the computing device 800 includes a processor 802 for controlling the functionality of the computing device 800. The processor 802 includes one or more circuits, such as general-purpose processors, special purpose processors, conventional processors, digital signal processors (DSPs), microprocessors, integrated circuits, a programmable logic device (PLD), application specific integrated circuits (ASICs), or the like. The processor 802 performs signal coding, data processing, power control, image processing, input/output processing, or any other functionality that enables the computing device 800 to perform as described herein.

The processor 802 stores information in or retrieve information from the memory 816. The memory 816 includes a non-removable memory or a removable memory. The non-removable memory includes random-access memory (RAM), read-only memory (ROM), a hard disk, or any other type of non-removable memory storage. The removable memory includes a subscriber identity module (SIM) card, a memory stick, a memory card (e.g., a digital camera memory card), or any other type of removable memory. The processor 802 accesses the memory 816 for executable instructions or other information that is used by the computing device 800. For example, the memory 816 may include computer-readable or machine-readable instructions that may be executed by the processor 802 for performing one or more methods, processes, or procedures, or portions thereof.

The computing device 800 includes a camera 806 that is in communication with the processor 802. The camera 806 is a digital camera or other optical device capable of generating images or videos (e.g., image sequences) for being captured at the computing device 800. The camera 806 includes a lighting device capable of flashing to in response to signals from the processor 802. The lighting device flashes to provide alerts via the camera 806.

The computing device 800 includes one or more communication circuits 818. The processor 802 is in electrical communication with the communication circuit 818 for sending or receiving information. The communication circuit 818 is capable of performing wired or wireless communications. For example, the communication circuit 818 includes one or more radio frequency (RF) transceivers for transmitting and receiving RF signals (e.g., BLUETOOTH®, near field communication (NFC), WIFI®, WI-MAX®, cellular, or other RF signals) via an antenna, or other communications module capable of performing wireless communications. In some embodiments, one or more communication circuits 818 are capable of performing infrared (IR) communications.

The processor 802 is in electrical communication with a keypad 824 for providing input to the processor 802. The keypad 824 includes one or more keys for receiving input from a user. The keypad 824 includes hard or soft keys for which the function of the keys changes as a user performs selections.

Other input into the processor 802 is provided by one or more sensors 826. The sensors 826 include a motion sensor, a proximity sensor, a heartrate monitoring sensor, an accelerometer, a gyroscope, or another sensor on the computing device. The motion sensor transmits infrared signals or uses image processing to sense movement. The proximity sensor transmits infrared signals to detect when an object is within a predefined proximity. The heartrate monitoring sensor implements photoplethysmography to detect the amount of blood flow in the user. The heartrate monitoring sensor includes one or more LED or photodiodes to detect the amount of blood flow in the user. The heartrate monitoring sensor implements infrared technology to detect the amount of blood flow in the user. The heartrate monitoring sensor takes an electrocardiogram (ECG) and detects information about the user's heartrate from the ECG. The accelerometer measures the non-gravitational acceleration of the computing device 800 in a given direction. The accelerometer responds to vibrations associated with movement in a given direction. The measurements from the accelerometer are used by the processor 802 to determine the magnitude or direction of the relative movement of the computing device 800, or the user's relative position (e.g., standing, sitting, or lying down). The gyroscope is used to determine the orientation of the computing device 800.

The processor 802 is in electrical communication with or generate images on a display 820 for providing information to a user. The communication between the display 820 and the processor 802 is a two-way communication, as the display 820 includes a touch screen module capable of receiving information from a user and providing such information to the processor 802. For example, the display 820 provides soft buttons for selection by a user that are recognized by the touch screen module and provided to the processor 802 as input.

The processor 802 is in electrical communication with or control a speaker 808. The speaker 808 provides an audible sound (e.g., tone, beep, or buzz) in response to a triggering event detected by the processor 802.

The computing device 800 includes an electric motor 810 that is in electrical communication with or controlled by the processor 802. The electric motor 810 rotates and causes the computing device 800 to vibrate (e.g., to indicate an alert) in response to a triggering event detected by the processor 802. The electric motor 810 provides an alert to supplement the audible alarm or replace the audible alarm provided by the speaker 808.

The processor 802 is in electrical communication with or receive information from a microphone 814. For example, the processor 802 receives audio signals via the microphone 814.

The computing device 800 includes a global positioning system (GPS) circuit 804. The GPS circuit 804 is capable of receiving GPS information. The processor 802 is capable of determining the GPS coordinates (e.g., latitude and longitude) of the computing device 800 based on the GPS information received via the GPS circuit.

The computing device 800 includes a visual indicator, such as one or more light-emitting diodes (LEDs) 812. In some embodiments, one or more LEDs 812 are illuminated or flashed to provide an alert or communicate other information to the user (e.g., low battery or turning on of the device)

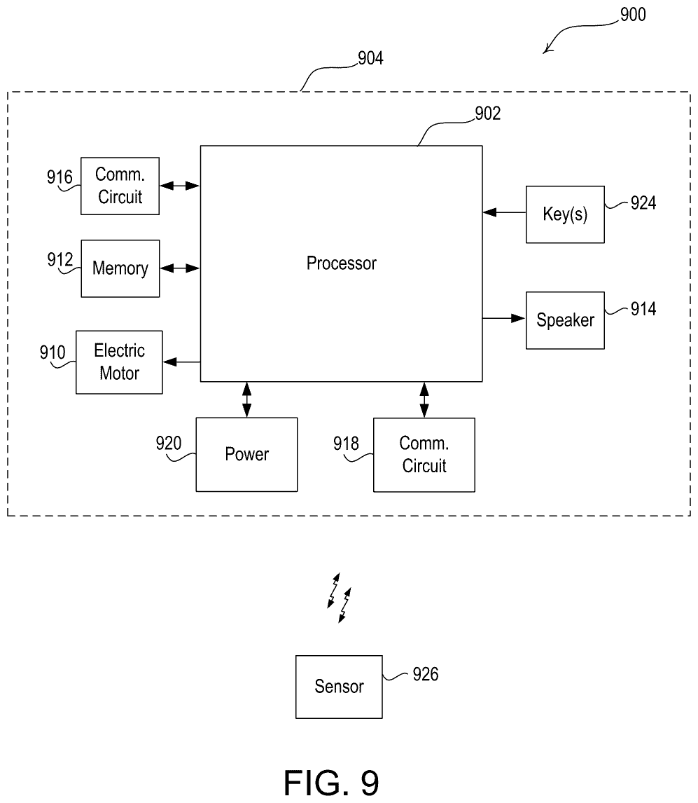

FIG. 9 is a block diagram of an example blood glucose monitoring device 900. In some embodiments, the blood glucose monitoring device 900 is a CGM or FGM, for example. The blood glucose monitoring device 900 includes a subcutaneous sensor 926 that is used to sense and monitor the amount of glucose in interstitial fluid of the user. Data is transmitted from the sensor 926 to a transmitting device 904. When the blood glucose monitoring device 900 is a CGM, the transmitting device 904 is located directly over the sensor 926 and wirelessly powers the data transfer from the sensor 926 via power supply 920. When the blood glucose monitoring device 900 is an FGM, the transmitting device 904 is a mobile device or other reader device that instantaneously receives the blood glucose information from the sensor 926 when the device is within the RF range of the sensor 926.

The transmitting device 904 receives data communications from the sensor 926 via a communication circuit 918. The communication circuit 918 is in electrical communication with a processor 902. The processor 902 includes one or more circuits, such as general-purpose processors, special purpose processors, conventional processors, digital signal processors (DSPs), microprocessors, integrated circuits, a programmable logic device (PLD), application specific integrated circuits (ASICs), or the like. The processor 902 performs signal coding, data processing, power control, input/output processing, or any other functionality that enables the transmitting device 904 to perform as described herein.

The transmitting device 904 includes another communication circuit 916 for communicating with other devices. The processor 902 is in electrical communication with the communication circuit 916 for sending or receiving information. The communication circuits 916, 918 are capable of performing wired or wireless communications. For example, the communication circuits 916, 918 include one or more radio frequency (RF) transceivers for transmitting and receiving RF signals (e.g., BLUETOOTH®, near field communication (NFC), WIFI®, WI-MAX®, cellular, or other RF signals) via an antenna, or other communications module capable of performing wireless communications. The communication circuits 916, 918 communicate using the same RF protocol or a different RF protocol.

The processor 902 stores information in or retrieves information from the memory 912. The memory 912 includes a non-removable memory or a removable memory. The non-removable memory includes random-access memory (RAM), read-only memory (ROM), a hard disk, or any other type of non-removable memory storage. The removable memory includes a subscriber identity module (SIM) card, a memory stick, a memory card (e.g., a digital camera memory card), or any other type of removable memory. For example, the memory 912 may include computer-readable or machine-readable instructions that may be executed by the processor 902 for performing one or more methods, processes, or procedures, or portions thereof. The processor 902 accesses the memory 912 for executable instructions or other information that is used by the transmitting device 904. The processor 902 is in electrical communication with a one or more input keys 924 for providing input to the processor 902.

The processor 902 is in electrical communication with or control a speaker 914. The speaker 914 provides an audible sound (e.g., tone, beep, or buzz) in response to a triggering event detected by the processor 902.

The blood glucose monitoring device 900 includes an electric motor 910 that is in electrical communication with or controlled by the processor 902. The electric motor 910 rotates and causes the blood glucose monitoring device 900 to vibrate (e.g., to indicate an alert) in response to a triggering event detected by the processor 902. The electric motor 910 provides an alert to supplement the audible alarm or replace the audible alarm provided by the speaker 914.

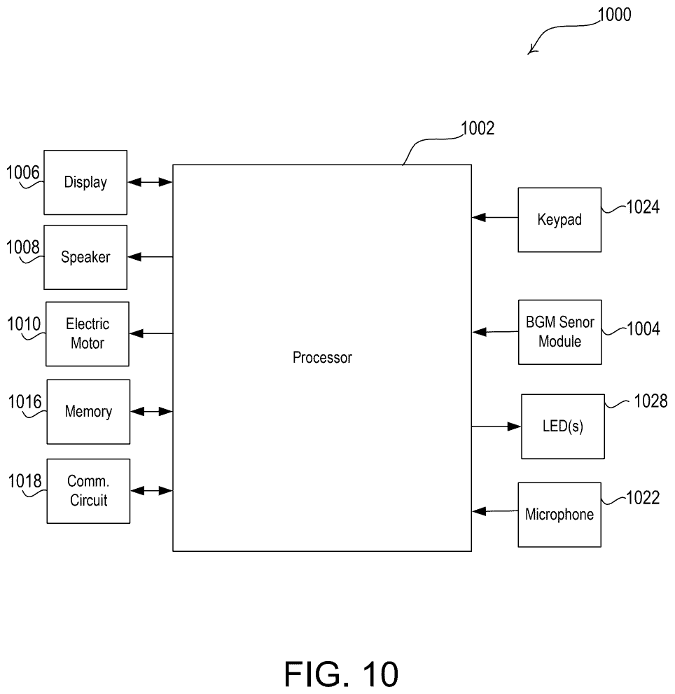

FIG. 10 is a block diagram of an example blood glucose meter (BGM) device 1000. As shown in FIG. 10, the BGM device 1000 includes a processor 1002 for controlling the functionality of the BGM device 1000. The processor 1002 includes one or more circuits, such as general-purpose processors, special purpose processors, conventional processors, digital signal processors (DSPs), microprocessors, integrated circuits, a programmable logic device (PLD), application specific integrated circuits (ASICs), or the like. The processor 1002 performs signal coding, data processing, power control, image processing, input/output processing, or any other functionality that enables the BGM device 1000 to perform as described herein.

The processor 1002 stores information in or retrieve information from the memory 1016. The memory 1016 includes a non-removable memory or a removable memory. The non-removable memory includes random-access memory (RAM), read-only memory (ROM), a hard disk, or any other type of non-removable memory storage. The removable memory includes a subscriber identity module (SIM) card, a memory stick, a memory card (e.g., a digital camera memory card), or any other type of removable memory. The memory 1016 may include computer-readable or machine-readable instructions that may be executed by the processor 1002 for performing one or more methods, processes, or procedures, or portions thereof. The processor 1002 accesses the memory 1016 for executable instructions or other information that is used by the BGM device 1000.

The BGM device 1000 includes one or more communication circuits 1018. The processor 1002 is in electrical communication with the communication circuit 1018 for sending or receiving information. The communication circuit 1018 is capable of performing wired or wireless communications. For example, the communication circuit 1018 includes one or more radio frequency (RF) transceivers for transmitting and receiving RF signals (e.g., BLUETOOTH®, near field communication (NFC), WIFI®, WI-MAX®, cellular, or other RF signals) via an antenna, or other communications module capable of performing wireless communications. In some embodiments, one or more communication circuits 1018 are capable of performing infrared (IR) communications.

The processor 1002 is in electrical communication with a keypad 1024 for providing input to the processor 1002. The keypad 1024 includes one or more keys for receiving input from a user. The keypad 1024 includes hard or soft keys for which the function of the keys changes as a user performs selections.

Other input into the processor 1002 is provided by the BGM sensor module 1004. The BGM sensor module 1004 includes a blood glucose measuring engine that may analyze blood samples provided by a patient on a blood glucose measurement strip and measures the amount of blood glucose in the samples.

The processor 1002 is in electrical communication with or generate images on a display 1006 for providing information to a user. The communication between the display 1006 and the processor 1002 is a two-way communication, as the display 1006 includes a touch screen module capable of receiving information from a user and providing such information to the processor 1002. For example, the display 1006 provides soft buttons for selection by a user that are recognized by the touch screen module and provided to the processor 1002 as input.

The processor 1002 is in electrical communication with or control a speaker 1008. The speaker 1008 provides an audible sound (e.g., tone, beep, or buzz) in response to a triggering event detected by the processor 1002.

The BGM device 1000 include an electric motor 1010 that is in electrical communication with or controlled by the processor 1002. The electric motor 1010 rotates and causes the BGM device 1000 to vibrate (e.g., to indicate an alert) in response to a triggering event detected by the processor 1002. The electric motor 1010 provides an alert to supplement the audible alarm or replace the audible alarm provided by the speaker 1008.

The processor 1002 is in electrical communication with or receive information from a microphone 1022. For example, the processor 1002 receives audio signals via the microphone 1022.

The BGM device 1000 includes a visual indicator, such as one or more one or more light-emitting diodes (LEDs) 1028. In some embodiments, one or more LEDs 1028 are illuminated or flashed to provide an alert or communicate other information to the user (e.g., low battery or turning on of the device).

Although features, elements, and functions are described above in particular combinations, a feature, element, or function is used alone or in any combination with the other features, elements, or functions. Various presently unforeseen or unanticipated alternatives, modifications, variations, or improvements may be subsequently made that are also intended to be encompassed by the following claims.

The methods described herein are implemented in a computer program, software, or firmware incorporated in a computer-readable medium for execution by a computer or processor. Examples of computer-readable media include electronic signals (transmitted over wired or wireless connections) and computer-readable storage media. Examples of computer-readable storage media include, but are not limited to, a read only memory (ROM), a random-access memory (RAM), removable disks, and optical media such as CD-ROM disks, and digital versatile disks (DVDs).

What is claimed is:

1. A method comprising:
determining an event of a therapeutic protocol;
detecting a triggering event for generating a notification associated with the event of the therapeutic protocol;
providing the notification to a user to assist in treatment of a medical condition in response to the detected triggering event associated with the event of the therapeutic protocol;
determining whether a response to the notification is received;
on a condition that the response to the notification is received, performing management of the treatment of the medical condition;
on a condition that the response to the notification fails to be received, contextualizing the failure to respond to the notification for the event of the therapeutic protocol based on contextual information that indicates an inability of the user to respond to the notification; and
adjusting a timer corresponding to the notification of the event of the therapeutic protocol based on the contextual information.

2. The method of claim 1, wherein the management of the treatment of the medical condition comprises a diabetic condition, and wherein the management of the treatment of the diabetic condition is performed by:
adjusting a scheduled time to monitor fasting blood glucose levels of the user,
modifying a therapeutic dose of the therapeutic protocol, or
adjusting a scheduled time to administer the therapeutic dose.

3. The method of claim 1, wherein the contextual information includes calendar information or location information associated with the mobile device, and wherein the contextualizing the failure to respond is based on the calendar information or the location information associated with a mobile device.

4. The method of claim 1, further comprising:
activating a triggering event detection timeframe associated with the event of the therapeutic protocol, wherein the triggering event is detected within the triggering event detection timeframe.

5. The method of claim 1, wherein detecting the triggering event further comprises:
detecting a keyword from at least one of a calendar event title or a calendar event location, wherein the calendar event title or the calendar event location are included in the contextual information; and
comparing the detected keyword to keywords associated with the notification; and
wherein the adjusting the timer corresponding to the notification is based on the comparison of the detected keyword and the keywords associated with the notification.

6. The method of claim 5, wherein the adjusting the timer corresponding to the notification comprises adjusting the timer of the notification to coincide with an occurrence of a calendar event associated with the at least one of the calendar event title or the calendar event location.

7. The method of claim 1, wherein detecting the triggering event further comprises:
detecting a keyword from at least one of a calendar event or location information associated with a mobile device associated with a user, wherein the calendar event title or the location information are included in the contextual information; and
comparing the detected keyword to keywords associated with the notification; and
wherein the adjusting the timer corresponding to the notification is based on the comparison of the detected keyword and the keywords associated with the notification.

8. The method of claim 1, wherein the therapeutic protocol comprises an insulin titration protocol.

9. The method of claim 8, wherein the event is a measurement event for measuring a fasting blood glucose level, the method further comprising:
determining that the fasting blood glucose level is within a targeted therapeutic range; and
wherein the providing the notification to the user is related to the fasting blood glucose level being within the targeted therapeutic range.

10. The method of claim 8, wherein the event is a dosing event for providing a dose of insulin, the method further comprising:
determining an optimal dose of insulin associated with the dosing event to achieve a targeted therapeutic range; and
wherein the notification comprises the optimal dose of insulin to achieve the targeted therapeutic range, and wherein the notification is configured to be displayed on a mobile device associated with the user.

11. The method of claim 1, wherein the triggering event comprises at least one of an activity that characterizes a circadian rhythm of the user, a device display triggering event, a device movement triggering event, a biometric triggering event, a time event, a period of time, a triggering event based on location information, or a triggering event based on calendar information, or triggering events based on location information and calendar information.

12. The method of claim 1, wherein the contextual information comprises one or more of calendar information, location information, an activity that characterizes a circadian rhythm of the user, or a biometric triggering event, and wherein the contextualizing the failure to respond is based on the one or more of calendar information, location information, an activity that characterizes a circadian rhythm of the user, or a biometric triggering event.

13. A method comprising:
determining an event of a therapeutic protocol;
detecting at least one triggering event for generating a notification associated with the event of the therapeutic protocol;
determining that the at least one triggering event exceeds a notification threshold indicating a level of confidence that a user will receive a notification; and
in response to the at least one triggering event exceeding the notification threshold, providing the notification to the user to assist in treatment of a medical condition.

14. The method of claim 13, further comprising adjusting the notification threshold to increase or decrease a level of confidence based on the at least one detected triggering event.

15. The method of claim 13, wherein the at least one triggering event is predetermined for the corresponding notification.

16. The method of claim 13, wherein the at least one triggering event comprises a first triggering event, the method further comprising detecting one or more additional triggering events to confirm that the notification should be provided.

17. The method of claim 13, wherein the at least one triggering event comprises a plurality of triggering events, and wherein each triggering event is associated with a corresponding weight for meeting the notification threshold.

18. The method of claim 17, further comprising:
determining a relative influence of each of the plurality of triggering events on obtaining a response to the notification; and
assign the corresponding weight to each triggering event of the plurality of triggering events based on the relative influence for obtaining the response to the notification.

19. The method of claim 13, further comprising:
determining whether a response to the notification is received;
on a condition that the response to the notification is received, performing management of the treatment of the medical condition; and
on a condition that the response to the notification fails to be received, contextualizing the failure to respond to the notification for the event of the therapeutic protocol.

20-25. (canceled)
